(19) United States
(12) Patent Application Publication
(10) Pub. No.: US 2022/0165375 A1
(43) Pub. Date: May 26, 2022

(54) METHODS, SYSTEMS, APPARATUSES, AND DEVICES FOR FACILITATING DELIVERING OF MEDICAL INFORMATION OF PATIENTS

(71) Applicants: Donald Henry French, SR., Las Vegas, NV (US); Donald Henry French, II, Acworth, GA (US)

(72) Inventors: Donald Henry French, SR., Las Vegas, NV (US); Donald Henry French, II, Acworth, GA (US)

(21) Appl. No.: 17/535,923

(22) Filed: Nov. 26, 2021

Related U.S. Application Data
(60) Provisional application No. 63/118,402, filed on Nov. 25, 2020.

(51) Int. Cl. G16H 10/60; G16H 40/67; G16H 40/20; G06F 16/93

(52) U.S. Cl. CPC G16H 10/60; G16H 40/67; G16H 15/00; G06F 16/93; G16H 40/20

(57) ABSTRACT

A method and system for facilitating delivery of medical information of patients is provided. Further, the method may include receiving a medical information of a patient from one or more source devices, analyzing this unprocessed version of the medical information using dynamic configurations, identifying one or more delivery devices and criteria, generating a delivery version of the medical information associated with the patient based on the unprocessed version of the medical information and the one or more delivery criteria and transmitting the delivery version of the medical information to the one or more delivery connectors which process the delivery version into a device specific version that the delivery device understands.

The current application claims a priority to the U.S. provisional patent application Ser. No. 63/118,402 filed on Nov. 25, 2020.

### FIELD OF THE INVENTION

Generally, the present disclosure relates to the field of data processing. More specifically, the present disclosure relates to methods, systems, apparatuses, and devices for facilitating delivering of medical information of patients.

### BACKGROUND OF THE INVENTION

In healthcare, each health-entity maintains a health record of a patient. Further, an entity requiring a patient's health records (Recipient/Requestor) may officially request a healthcare-entity (or Information Owner (IO)) to provide health information of the patient that may be maintained by the IO. Further, the Requestor needs the health record regarding the patient's visit to the Emergency room or admission to a local hospital or one across the country to provide proper healthcare to the patient. Further, to receive the health information, the Requestor submits a Request of Information (ROI) to the second health entity for the health information needed. Upon receiving the ROI from the Requestor, the IO pulls the requested health information from their medical record system and sends the requested health information to the Recipient.

Currently, health information is held by various health entities like physician practices, hospitals, labs, and other clinical, diagnostic or treatment centers—healthcare providers. To provide healthcare today, physicians need information from wherever it is stored that is normally done with signed Release of Information requests (ROI). The problem is that office-based healthcare providers are standalone entities in most cases and do not have direct access to the information as hospital-based physicians do. Even today, most responses to ROI requests are faxed, emailed, or printed. Further, the documents are usually in a large image (PDF/FAX) file. The Recipient must then split the file into new separate files for each document contained in the request and, in the received file. Further, the Recipients that are using EMRs need to get the information into it, this is done via scanning the received documents and indexing them in to the proper patients record and type of document. Further, the scanning process is manually intensive and expensive. Further, the Recipient may be connected to the sending IO as one of their affiliated practices and the IO may place the documents directly in the practice's EMR. This is very efficient, though usually only available to larger hospital entities and their office-based physicians. Furthermore, the current technologies do not securely and confidentially exchange the requested documents between the IO and Recipient.

Therefore, there is a need for methods, systems, apparatuses, and devices for facilitating delivering of medical information of patients that may overcome one or more of the above-mentioned problems and/or limitations.

### BRIEF SUMMARY OF THE INVENTION

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope.

Disclosed herein is a method for facilitating delivering of medical information of patients to a requestor's device, in accordance with some embodiments. Accordingly, the method may include a step of receiving, using a communication device, at least one request comprising at least one medical information of at least one patient from at least one source device. Further, the method may include a step of analyzing, using a processing device, the at least one request using at least one defined criteria of the implementation. Further, the method may include a step of identifying, using the processing device, at least one destination device based on the analyzing of the at least one request, wherein the identifying of the at least one destination device comprises identifying at least one delivery device. Further, the method may include a step of determining, using the processing device, at least one delivery criterion based on the analyzing of the at least one request. Further, the method may include a step of generating, using the processing device, an updated request of the at least one medical information associated with the at least one patient based on the unprocessed request of the at least one medical information and the at least one delivery criterion, wherein the updated request comprises a processed request. Further, the method may include a step of transmitting, using the communication device, the processed request of the at least one medical information to the at least one destination device. Further, the method may include a step of receiving, using the communication device, at least one recipient information comprising demographics and the at least one delivery device from the at least one source device. Further, the method may include a step of storing, using a storage device, a validated version of the at least one recipient information in a database. Further, the method may include a step of receiving, using the communication device, at least one medical information for patients with at least one recipient from the at least one source device. Further, the method may include a step determining which of the at least one recipient would like to receive the one medical information by validating their choice in a database. Further, the method may include a step of generating using the processing device, a request containing the at least one medical information and at least one destination.

Further, disclosed herein is a method for facilitating delivering of medical information of patients, in accordance with some embodiments. The method may include a step of receiving, using a communication device, one or more ROIs (Release Of Information) requests for receiving one or more medical information of one or more patients from one or more source devices. Further, the method may include a step of analyzing, using a processing device, the one or more ROIs using one or more dynamic configurations. Further, the method may include a step of identifying, using the processing device, one or more delivery devices based on the analyzing of the one or more ROIs. Further, the method may include a step of determining, using the processing device, one or more delivery criteria based on the analyzing of the one or more ROIs. Further, the method may include a step of generating, using the processing device, a delivery version of the one or more medical information associated with the one or more patients based on the unprocessed version of the one or more medical information and the one or more delivery criteria. Further, the method may include a step of converting, using the processing device, the delivery version of the one or more medical information into a specific device type version of the one or more medical information using one or more dynamic profiles. Further, the method may include a step of transmitting, using the communication device, the specific device type version of the one or more medical information to the one or more delivery devices.

Further disclosed herein is a system for facilitating delivering of medical information of patients, in accordance with some embodiments. The system may include a communication device and a processing device. Further, the communication device may be configured for performing a step of receiving one or more types of ingresses for receiving one or more medical information of one or more patients from one or more source devices. Further, the communication device may be configured for performing a step of transmitting a delivery device specific version of the one or more medical information to the one or more delivery devices. The processing device may be communicatively coupled with the communication device. Further, the processing device may be configured for performing a step of analyzing the one or more types of ingresses using one or more dynamic configurations. Further, the processing device may be configured for performing a step of identifying the one or more delivery devices based on the analyzing of the one or more types of ingresses. Further, the processing device may be configured for performing a step of determining one or more delivery criteria based on the analyzing of the one or more types of ingresses. Further, the processing device may be configured for performing a step of generating the device specific delivery version of the one or more medical information associated with the one or more patients based on the unprocessed version of the one or more medical information and the one or more delivery criteria.

Further disclosed herein is a method for facilitating delivering of medical information of patients to a requestor's device, in accordance with some embodiments. Accordingly, the method may include a step of receiving, using a communication device, at least one request comprising at least one medical information of at least one patient from at least one source device. Further, the method may include a step of analyzing, using a processing device, the at least one request using at least one defined criteria of the implementation. Further, the method may include a step of identifying, using the processing device, at least one destination device based on the analyzing of the at least one request, wherein the identifying of the at least one destination device comprises identifying at least one delivery device. Further, the method may include a step of receiving, using the communication device, an unprocessed request of the at least one medical information associated with the at least one patient from the at least one source device. Further, the method may include a step of determining, using the processing device, at least one delivery criterion based on the analyzing of the at least one request. Further, the method may include a step of generating, using the processing device, an updated request of the at least one medical information associated with the at least one patient based on the unprocessed request of the at least one medical information and the at least one delivery criterion, wherein the updated request comprises an interim request. Further, the method may include a step of transmitting, using the communication device, the interim request of the at least one medical information to a connector. Further, the method may include a step of receiving, using the communication device, at least one recipient information comprising demographics and the at least one delivery device from the at least one source device. Further, the method may include a step of storing, using a storage device, a validated version of the at least one recipient information in a database.

Further disclosed herein is a method for facilitating delivering of medical information of patients, in accordance with some embodiments. Accordingly, the method may include a step of receiving, using a communication device, an ingress connection for receiving a medical information of a patient from a source device. Further, the method may include a step of receiving, using the communication device, an unprocessed version of the medical information associated with the patient from the source device using the ingress connection. Further, the method may include a step of analyzing, using a processing device, the unprocessed version of the medical information using at least one dynamic configuration. Further, the method may include a step of identifying, using the processing device, at least one delivery device based on the analyzing of the unprocessed version of the medical information. Further, the method may include a step of generating, using the processing device, a delivery version of the medical information associated with the patient based on the unprocessed version of the medical information. Further, the method may include a step of transmitting, using the communication device, the delivery version of the medical information to a connector, wherein the connector is configured for creating a device specific version of the medical information based on the delivery version of the medical information for delivering the device specific version of the medical information to a specific type of the at least one delivery device.

Further disclosed herein is a system for facilitating delivering of medical information of patients, in accordance with some embodiments. Accordingly, the system may include a communication device and a processing device. Further, the communication device may be configured for receiving an ingress connection for receiving a medical information of a patient from a source device. Further, the communication device may be configured for receiving an unprocessed version of the medical information associated with the patient from the source device using the ingress connection. Further, the communication device may be configured for transmitting a delivery version of the medical information to a connector, wherein the connector is configured for creating a device specific version of the medical information based on the delivery version of the medical information for delivering the device specific version of the medical information to a specific type of the at least one delivery device. Further, the processing device may be communicatively coupled with the communication device. Further, the processing device is configured for analyzing the unprocessed version of the medical information using at least one dynamic configuration. Further, the processing device is configured for identifying at least one delivery device based on the analyzing of the unprocessed version of the medical information. Further, the processing device is configured for generating the delivery version of the medical information associated with the patient based on the unprocessed version of the medical information.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 is an illustration of an online platform consistent with various embodiments of the present disclosure.

FIG. 2 is a block diagram of a computing device for implementing the methods disclosed herein, in accordance with some embodiments.

FIG. 3 is a flowchart of a method for facilitating delivering of medical information of patients, in accordance with some embodiments.

FIG. 4 is a continuation flowchart of FIG. 3.

FIG. 5 is a flowchart of a method for facilitating delivering of medical information of patients, in accordance with further embodiments.

FIG. 6 is a flowchart of a method for facilitating delivering of medical information of patients, in accordance with further embodiments.

FIG. 7 is a continuation flowchart of FIG. 6.

FIG. 8 is a flowchart of a method for facilitating delivering of medical information of patients, in accordance with further embodiments.

FIG. 9 is a flowchart of a method for facilitating delivering of medical information of patients, in accordance with further embodiments.

FIG. 10 is a continuation flowchart of FIG. 9.

FIG. 11 is a flowchart of a method for facilitating delivering of medical information of patients, in accordance with further embodiments.

FIG. 12 is a continuation flowchart of FIG. 11.

FIG. 13 is a flowchart of a method for facilitating delivering of medical information of patients, in accordance with further embodiments.

FIG. 14 is a flowchart of a method for facilitating delivering of medical information of patients, in accordance with further embodiments.

FIG. 15 is a flowchart of a method for facilitating delivering of medical information of patients. in accordance with further embodiments.

FIG. 16 is a block diagram of a system for facilitating delivering of medical information of patients, in accordance with some embodiments.

FIG. 17 is a block diagram of the system for facilitating delivering of medical information of patients, in accordance with further embodiments.

FIG. 18 is a block diagram of a system for facilitating the exchange of data, especially medical data associated with a patient, between a Healthcare entity possessing the data and a Recipient in accordance with some embodiments.

FIG. 19 is a flow diagram of a general delivery method using a GRPC protocol and buffers, in accordance with some embodiments.

FIG. 20 is a flow diagram related to a basic implementation of a message queue ingress, in accordance with some embodiments.

FIG. 21 is a flow diagram associated with the Gateway Controller, in accordance with some embodiments.

FIG. 22 is a flow diagram associated with the Delivery Controller, in accordance with some embodiments.

FIG. 23 is a flowchart of a method for facilitating delivering of medical information of patients to a requestor's device, in accordance with some embodiments.

FIG. 24 is a block diagram of a system for facilitating the exchange of data, especially medical data associated with a patient, in accordance with some embodiments.

FIG. 25 is a flowchart of a method for automatic delivery of ROI, in accordance with some embodiments.

FIG. 26 is a block diagram of a system showing the basic functionality of a connector used for pluggable ingress and final device delivery processes, in accordance with some embodiments.

### DETAILED DESCRIPTION OF THE INVENTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim limitation found herein and/or issuing here from that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings and specifications. They are divided into three sections. 1) Manual ROI Request, 2) Automatic Document Delivery, 3) HIPAA Validation. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the claims found herein and/or issuing here from. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of facilitating delivering of medical information of patients, embodiments of the present disclosure are not limited to use only in this context.

In general, the method disclosed herein may be performed by one or more computing devices. For example, in some embodiments, the method may be performed by a server computer in communication with one or more client devices over a communication network such as, for example, the Internet. In some other embodiments, the method may be performed by one or more of at least one server computer, at least one client device, at least one network device, at least one sensor, and at least one actuator. In some other embodiments, the method may be performed by one or more of at least one server computer each running containers processing the various processes define in the embodiment. Examples of the one or more client devices and/or the server computer may include, a desktop computer, a laptop computer, a tablet computer, a personal digital assistant, a portable electronic device, a wearable computer, a smart phone, an Internet of Things (IoT) device, a smart electrical appliance, a video game console, a rack server, a super-computer, a mainframe computer, mini-computer, micro-computer, a storage server, an application server (e.g., a mail server, a web server, a real-time communication server, an FTP server, a virtual server, a proxy server, a DNS server, etc.), a quantum computer, and so on. Further, one or more client devices and/or the server computer may be configured for executing a software application such as, for example, but not limited to, an operating system (e.g., Windows, Mac OS, Unix, Linux, Android, etc.) in order to provide a user interface (e.g., GUI, touch-screen based interface, voice based interface, gesture based interface, etc.) for use by the one or more users and/or a network interface for communicating with other devices over a communication network. Accordingly, the server computer may include a processing device configured for performing data processing tasks such as, for example, but not limited to, analyzing, identifying, determining, generating, transforming, calculating, computing, compressing, decompressing, encrypting, decrypting, scrambling, splitting, merging, interpolating, extrapolating, redacting, anonymizing, encoding, and decoding. Further, the server computer may include a communication device configured for communicating with one or more external devices. The one or more external devices may include, for example, but are not limited to, a client device, a third-party database, a public database, a private database, and so on. Further, the communication device may be configured for communicating with the one or more external devices over one or more communication channels. Further, the one or more communication channels may include a wireless communication channel and/or a wired communication channel. Accordingly, the communication device may be configured for performing one or more of transmitting and receiving of information in electronic form. Further, the server computer may include a storage device configured for performing data storage and/or data retrieval operations. In general, the storage device may be configured for providing reliable storage of digital information. Accordingly, in some embodiments, the storage device may be based on technologies such as, but not limited to, data compression, data backup, data redundancy, deduplication, error correction, data fingerprinting, role-based access control, and so on.

Further, one or more steps of the method disclosed herein may be initiated, maintained, controlled, and/or terminated based on a input received from one or more systems operated by one or more Information Owner. Further, the user as defined herein may refer to a human, an animal, or an artificially intelligent being in any state of existence, unless stated otherwise, elsewhere in the present disclosure. Further, in some embodiments, the one or more users may be required to successfully perform authentication in order for the control input to be effective. In general, a user of the one or more users may perform authentication based on the possession of a secret human readable secret data (e.g., username, password, passphrase, PIN, secret question, secret answer, etc.) and/or possession of a machine readable secret data (e.g., encryption key, decryption key, bar codes, etc.) and/or or possession of one or more embodied characteristics unique to the user (e.g., biometric variables such as but not limited to, fingerprint, palm-print, voice characteristics, behavioral characteristics, facial features, iris pattern, heart rate variability, evoked potentials, brain waves, and so on) and/or possession of a unique device (e.g., a device with a unique physical and/or chemical and/or biological characteristic, a hardware device with a unique serial number, a network device with a unique IP/MAC address, a telephone with a unique phone number, a smartcard with an authentication token stored thereupon, etc.). Accordingly, the one or more steps of the method may include communicating (e.g., transmitting and/or receiving) with one or more sensor devices and/or one or more actuators in order to perform authentication. For example, the one or more steps may include receiving, using the communication device, the secret human readable data from an input device such as, for example, a keyboard, a keypad, a touch-screen, a microphone, a camera, and so on. Likewise, the one or more steps may include receiving, using the communication device, the one or more embodied characteristics from one or more biometric sensors.

Further, one or more steps of the method may be automatically initiated, maintained, and/or terminated based on one or more predefined conditions. In an instance, the one or more predefined conditions may be based on one or more contextual variables. In general, the one or more contextual variables may represent a condition relevant to the performance of the one or more steps of the method. The one or more contextual variables may include, for example, but are not limited to, location, time, identity of a user associated with a device (e.g., the server computer, a client device, etc.) corresponding to the performance of the one or more steps, environmental variables (e.g., temperature, humidity, pressure, wind speed, lighting, sound, etc.) associated with a device corresponding to the performance of the one or more steps, physical state and/or physiological state and/or psychological state of the user, physical state (e.g., motion, direction of motion, orientation, speed, velocity, acceleration, trajectory, etc.) of the device corresponding to the performance of the one or more steps and/or semantic content of data associated with the one or more users. Accordingly, the one or more steps may include communicating with one or more sensors and/or one or more actuators associated with the one or more contextual variables. For example, the one or more sensors may include, but are not limited to, a timing device (e.g., a real-time clock), a location sensor (e.g., a GPS receiver, a GLONASS receiver, an indoor location sensor, etc.), a biometric sensor (e.g., a fingerprint sensor), an environmental variable sensor (e.g., temperature sensor, humidity sensor, pressure sensor, etc.) and a device state sensor (e.g., a power sensor, a voltage/current sensor, a switch-state sensor, a usage sensor, etc. associated with the device corresponding to performance of the or more steps).

Further, the one or more steps of the method may be performed one or more number of times. Additionally, the one or more steps may be performed in any order other than as exemplarily disclosed herein, unless explicitly stated otherwise, elsewhere in the present disclosure. Further, two or more steps of the one or more steps may, in some embodiments, be simultaneously performed, at least in part. Further, in some embodiments, there may be one or more time gaps between performance of any two steps of the one or more steps.

### Overview

According to some embodiments, the present disclosure relates to methods and systems for facilitating the exchange of data, especially medical data associated with a Patient between a Healthcare-entity (IO—Information Owner) and any requestor.

According to some embodiments, a system for facilitating the exchange of data, especially medical data for a patient between healthcare providers is disclosed. Accordingly, the system may include a communication device, a processing device, and a storage device.

According to some aspects, a method for facilitating the exchange of data, especially medical data associated with a patient to the patient is disclosed. Accordingly, the method may include receiving, using a communication device, a request from at least one source device. Further, the method may include analyzing, using a processing device, the request to generate a delivery of medical data directly to the requestors' Personal Health Record (PHR). Further, the method may include transmitting, using the communication device, the requested medical data to at least one healthcare entity device. Further, the method may include receiving, using the communication device, the medical data in one data format from the at least one healthcare entity device. Further, the method may include processing, using the processing device, the medical data to generate processed medical data corresponding to another data format. Further, the method may include transmitting, using the communication device, the processed medical data to at least one requestor device. Further, the method may include transmitting, using the communication device, the processed medical data to at least one requestor device running one of the many EMRs on the market. Further, the method may include receiving, using the communication device, a delivery receipt from at least one requestor device. Further, the method may include storing, using a storage device, at least one of the medical data and the request.

According to some aspects, a system for facilitating the exchange of data, especially medical data associated with a patient between an IO and an entity requiring such information is disclosed. Accordingly, the system may allow an IO to connect to a multitude of EMR systems and deliver a patient's health record securely and confidentially directly into their EMR is disclosed.

According to some aspects, the present disclosure describes methods and systems for facilitating the exchange of data, especially medical data associated with a patient from a Requestor. In healthcare, each health-entity maintains a health record of a patient and must officially request a second health-entity to provide the health information the second health entity maintains for the same patient that the first health-entity requires to properly treat the patient. For example, the Primary Care Provider (Requestor), needs information regarding the patient's visit to the Emergency room or admission to a local hospital or one across the country (Information Owner or IO). Further, to receive the information, the Requestor may submit a Request Of Information (ROI) to the IO for the information required. Once the IO receives the request, the IO may pull the requested records associated with the ROI and package the records up to send to the requestor. Today this entails using the IOs Electronic Medical Record (EMR) and selecting the proper patient, selecting the requested documents, and somehow sending the information via fax, email, or print. The invention allows the easy pluggable addition of new delivery mechanisms using Connectors to deliver directly to the requestor's EMR (delivery mechanisms very greatly when delivering to each brand and model of EMR). When the requestor receives the information via a non EMR/electronic repository, the requestor may print and scan each document and put the second or third generation image in their EMR, or using direct EMR delivery it will be automatically placed in their EMR directly with no additional processing.

If the requestor is directly associated with or owned by or affiliated with the IO, the requestor, using the same brand of EMR as the IO, or a directly interfaced one, may electronically access the documents, directly or download and add them to their own EMR. The problem with this approach is what happens to the 90% plus of the other Requestors that are independent or owned by competitive IOs. These requestors must follow the request, print, scan approach.

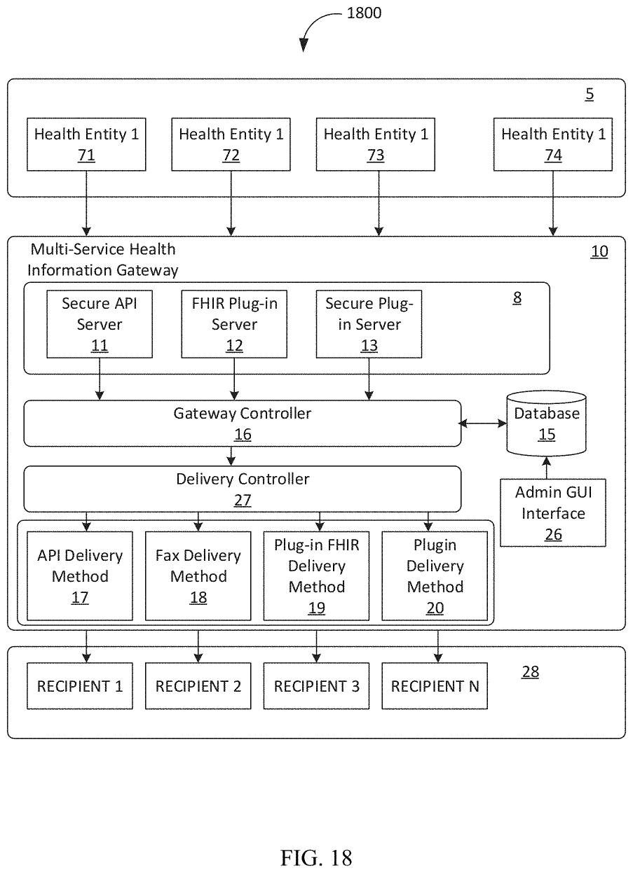

There are several other requestors of health information that are not health providers, for example, courts, insurance companies, attorneys, and patients. The Intelligent Health Information Gateway (IHIG) is one embodiment of the disclosed system. Further, the IOs using the IHIG may not need to change their current policies and practices. Further, the IOs may still receive the ROI from the requestor as normal, select the required documents, and instead of faxing or emailing them, they just submit the request through their interface to the IHIG. Further, the IHIG determines how the requestor desires to receive the information (any of the methods shown in items 17, 18, 19, 20 in FIG. 18 below. There is no manual intervention to determine the proper delivery process or preparation of documents to be delivered. As new delivery methods are added, for example into a Court's Document Management System item 20 in FIG. 18 below, the requests may be automatically added to that destination, at the Court's requirements. Further, the IOs using the IHIG may be able to reduce their manual ROI requests to change their current policies and practices using the Automatic Delivery process in IHIG. Automatic Delivery normally delivers one medical document to multiple physicians, real time, as soon as the document is completed in the hospital. Instead of ROI delivering multiple documents to one physician at the request of the physician.

Further, all IOs MUST always be very careful that every ROI is properly approved and only the requested information is provided. If not, they violate the Health Insurance Portability and Accountability Act (HIPPA), specifically, the security and privacy portion. Further, the transmission of any Personally Identifiable Information (PII) or Personal Health Information (PHI) electronically instead of via fax/paper may also be based on HIPPA. The security and privacy section defines the PHI and PII that may be delivered to a requestor and how the ROI may be handled.

Starting several years ago, there has been a growth in the Integrated Heath Exchange (IHE) in communities, counties, and states. These have not been very successful since patients are concerned as to who can get access to their PHI/PII. In most of the IHEs, any health provider may get access to any patient's record by just doing so. Further, existing technologies do not include an automated method to validate a valid reason for access. This is the reason that the option in these IHEs to limit remote access to emergency care only is normally selected by the patient if they join.

Further, the existing technologies may not solve the ROI problems on either the IO or the requestor side. The IHIG solves the problem on both sides. Once the IHIG is deployed at an IO, it is transparent to the normal operations of the IO's ROI process and gives the IO the ability to deliver PHI using means other than the current email, fax, or print. On the requestor's side, the IHIG allows the requestor to determine the mode the requestor may like to use in receiving the requested information. If the requestor is currently receiving the information via fax or email, the only thing they must do is respond to a verification fax/email to confirm the information the IO has is correct and meets HIPAA requirements. Other than that, the requestor may continue to receive information the same way they always have. If the requestor chooses to receive the information directly in their EMR or other methods, the requestor may have to sign up for those options and provide EMR login credentials connection information. Further, the IO may accept the ROI and may select the requestor from their current database of requestors or add to it, adding or modifying a requestor in the IO's database notifies IHIG of the new information and IHIG does all the HIPAA validation, selecting the information to be delivered and send it. In other words, the IHIG requestor database (20 in FIG. 18) is built automatically, and the IO may use their private list of requestors or share a larger area one built as Requestors are added to other IOs.

Further, The Intelligent Health Information Gateway (IHIG) allows the IO to submit the release into the IHIG specifying any recipient without specifying a delivery device/method. The release will be delivered how, when, and where the requestor desires based upon their configured delivery devices. The examples of delivery options are the standard Fax, Email, and Print along with the ability to deliver to various EMR such as AllScripts, Cerner, Epic, Athena plus more, with no human intervention, all using a pluggable system. Pluggable delivery connectors may be written in any language for custom destinations such as insurance companies and release services. The Intelligent Health Information Gateway solves this by providing one consistent method and point of contact for the Requestor to specify any delivery methods they have available. The IO uses this information to deliver the requested information as specified by the Requestor using their normal ROI processes.

Further, the disclosed system allows healthcare providers, private practices and hospitals, a simple method of connecting to a multitude of other healthcare providers, private practices, and hospitals, and optionally others such as a patients into their PHR to anyone who needs and is authorized to receive a copy of a patient's medical record securely and confidentially.

Further, the disclosed system provides a plurality of connection methods allowing healthcare providers to connect to the proper recipient's EMR or other desired method of receiving the ROI (fax, email, etc.). To receive anything other than Fax, Email, Print the recipient must provide credentials for the service and provide connection information. IHIG's Gateway controller (16) of FIG. 18 puts the information into a common format and each delivery module (17, 18, 19, 20) of FIG. 18 converts it into the proper format for the recipient.

Further, the disclosed system may provide a consistent common connection point for a healthcare provider to connect to multiple other requesting healthcare providers delivering a patient's record securely and confidentially using the data format and protocol the Recipient requires.

Once the IHIG is deployed at an IO, it is transparent to the normal operations of the IO's ROI process and gives the IO the ability to deliver PHI using means other than the current EMAIL, FAX, or PRINT. On the Requestor's side, it allows them to determine the method they would like to use in receiving the requested information. If the Requestor is currently receiving the information via FAX or EMAIL, the only thing they must do is respond to a verification FAX/EMAIL to confirm the information the IO has is correct and meets HIPAA requirements. Other than that, they will continue to receive information the same way they always have. If they would like to take advantage of the ability to receive the information directly in their EMR or other methods, they must provide credentials and connection information. On the IO side, the process is easier than it was. They accept the ROI request and do whatever they have done with it in the past. Then select the requestor from their current Database of requestors or add to it, select the information to be delivered and send it. IHIG does all the rest for EVERY requestor, not just those who have relationships with the IO.

The disclosure relates to an information distribution system operative for delivering a multitude of medical reports from an Information Owner (Hospital, Doctor's office, . . . ) by receiving a set of information from the Information Owner's EMR containing the destination (Requestor), a method of delivery and a multitude of reports to be delivered to a Requestor (Another hospital, Doctor's office, Patient (PHR), . . . ).

Further, the disclosed system may include a microcomputer-based controller having a network interface that is connected to the IO's EMR. The EMR submits a completed release to one of the ingress controllers depending on the EMR's required protocol. The controller is automatically operative to queue the documents in the release to a delivery queue which manages the delivery workload, for transmission to the intended recipient, utilizing a facsimile machine, fax board, other data communications device connected to the controller, or internet based FAX and EMAIL delivery services.

According to some embodiments, a system delivering medical information using a multitude of delivery methods (Other EMR's, Image repositories, Fax, Printers, Email, etc.) is disclosed.

According to some embodiments, the present disclosure provides a method for distributing requested Releases of Information generated by an EMR or other electronic systems, EMRs, Electronic repositories. Further, the method may include steps in receiving the Release of Information to be delivered, determining the delivery method, queuing it for delivery how, where, and when the Requestor desires, and the delivery of that Release to the proper destination.

Advantageously, the present disclosure saves the Recipient the time and money required to process the release delivered via paper. Besides delivery to a Recipient's EMR, the present disclosure also includes the standard Fax, Email, Print, and Batch Printing making it easily replace existing delivery systems in IO's current EMR.

According to some embodiments, a system for facilitating the exchange of data, especially medical data for a patient between healthcare providers is disclosed.

According to some aspects, a method for facilitating the exchange of data, especially medical data associated with a patient is disclosed. Accordingly, the method may include receiving, using a communication device, a release generated by an EMR or another electronic device.

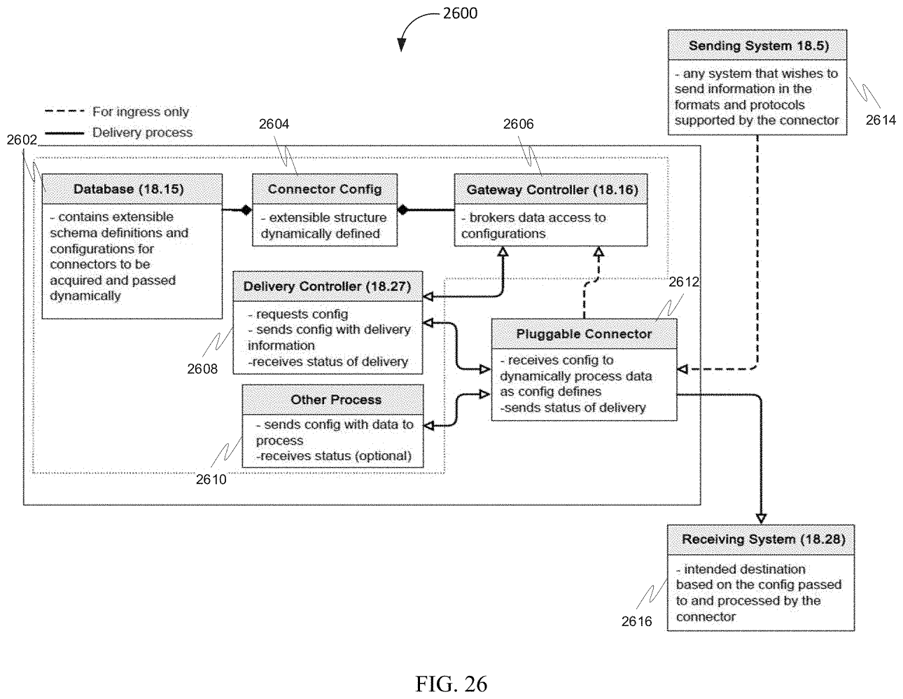

Further, the method may include transmitting, using the communication device, the release to at least one healthcare entity device. Further, the method may include receiving, using the communication device, the medical data in one data format from the at least one healthcare entity device. Further, the method may include processing, using the processing device, the medical data, and a conversion profile in a Connector FIG. 26 to generate processed medical data corresponding to another data format at the time of delivery. Further, the method may include transmitting, using the communication device, the processed medical data to at least one Recipient device. Further, the method may include receiving, using the communication device, a delivery receipt from at least one Recipient device.

Accordingly, the system may allow healthcare entities to connect to a multitude of Recipients, using different EMRs, and deliver a patient's health record securely and confidentially directly into the selected Recipient's EMR is disclosed.

Further, the method may include storing, using a storage device, at least one of the medical data and the request.

Further, the present disclosure describes methods and systems for facilitating the exchange of data, especially medical data associated with a patient. In healthcare, each health-entity (Recipient), the entity that requests and receives the data, maintains the health record of a patient of all care that entity as performed or received from others and must officially request a second health-entity (Information Owner or IO), to provide the Health Information (HI) they maintain for the same patient. For example, the Primary Care Provider (Recipient), needs HI regarding the patient's visit to the Emergency Room at a health-entity anywhere in the country, the IO. Further, to receive the HI, the Recipient may submit a Request of Information (ROI) to the IO specifying the required HI. Once the IO receives the ROI, the IO may pull/select the HI associated with the ROI and package it to send to the Recipient. Today this normally entails using the IOs Electronic Medical Record (EMR) and selecting the proper patient, selecting the requested HI, and somehow sending the information via fax or email. Further, the disclosure allows the easy pluggable addition of both ingress mechanisms and delivery mechanisms. (Selecting the delivery method and the delivery process and protocol varies between all EMRS) to the Recipient. For non-EMR delivery, when the Recipient receives the information, usually in paper form, they may print and scan each page of the HI and put the second or third generation lower quality image in their EMR, or in a very limited number of cases, it will be automatically placed in their EMR by their partner IOs.

If the Recipient is directly associated with, owned by, or affiliated with the IO, the Recipient, using the same brand of EMR as the IO, or a directly interfaced one, may electronically access the HI, directly download, and add them to their own EMR. The problem with this approach is what happens to the 90% plus of the Recipients that are independent of the IO. These Recipients must follow the request, print, scan, and insert approach.

There are several other types of entities that require and receive health information that is not health providers, for example, courts, insurance companies, attorneys, and patients. These requested HI is normally delivered via email, fax, or print and mail. They must follow the time consuming and costly request, print for those that maintain paper records, and insert for those that have electronic repositories. Further, an IO using the present disclosure may not need to change their current policies and practices to handle any type of Recipient or delivery mechanism. Further, the IO may still receive the ROI from the Recipient, select the required documents, and instead of using their current faxing or emailing system, they submit the HI set through their interface to the present disclosure (item 5 in FIG. 18). Further, the present disclosure determines how the Recipient desires to receive the information (any of the methods shown in items 17, 18, 19, 20 (referring to FIG. 18)) from the profile they manage. There is no manual intervention to determine the proper delivery process or preparation of documents to be delivered. As new delivery methods are added, for example into a Court's Document Management System (item 20 in FIG. 18), the HI may be automatically added to that destination, at the Court's requirements.

Further, all IOs MUST always be very careful that every ROI is properly approved and only the requested HI is provided. If not, they violate the Health Insurance Portability and Accountability Act (HIPPA), specifically, the security and privacy portion. Further, the transmission of any Personally Identifiable Information (PII) or Personal Health Information (PHI) electronically instead of via fax/paper may also be based on HIPPA rules. The security and privacy section defines the PHI and PII that may be delivered to a Recipient and how the ROI may be handled. The present disclosure assists in this by verifying that each Document in the HI set is delivered to the Recipient at the Recipients verified destination (VERIFICATION PROCESS) or both the Recipient and the IO are notified of failures.

Starting several years ago, there has been a growth in the Integrated Heath Exchange (IHE) in communities, counties, and states. These have not been very successful since patients are concerned as to who has access to their PHI/PII. In most of the IHEs, any health provider may get access to any patient's record by just doing so. Further, existing technologies do not include an automated method to validate a valid reason for access. This is the reason that the option in these IHEs to limit remote access to emergency care only is normally selected by the patient.

Further, the existing technologies may not solve the ROI problems on either the IO or the Recipient side. The present disclosure solves the ROI problems on both sides. Once the present disclosure is deployed for an IO, it is transparent to the normal operations of the IO's ROI process and gives the IO the ability to deliver PHI/PPI securely using means other than the current email, fax, or print which are normally not secure. On the Recipient's side, the present disclosure allows the Recipient to determine the method they may like to use in receiving the requested HI. If the Recipient has defined multiple methods of delivery, they may specify their desired validate delivery method. If none is specified, the delivery is made via the device defined as primary in their profile. If the Recipient is currently receiving the information via fax, print, or email and wishes to continue, the only thing they must do is respond to a verification fax/email sent to confirm the information the IO has is correct and meets HIPAA requirements. This validation process automatically occurs multiple times per year as specified by the IO. Other than that, the Recipient may continue to receive information the same way they always have. If the Recipient chooses to receive the HI directly in their EMR or other electronic methods, the Recipient may have to sign up for those options. Further, the IO may accept the ROI and may select the Recipient from their current database of Recipients or add to it, select the HI to be delivered and send it. In other words, the Recipient database is built automatically, and the IO may use their private list of Recipients or share a larger area one built as Recipients are added to other IOs. Noting is delivered to a device without the device being verified, see Validation Process.

Further, the present disclosure allows the IO to submit the release into it specifying any valid Recipient. The release will be delivered how, when, and where the Recipient desires. The examples of delivery options are the standard Fax, Email, and Print along with the ability to deliver to various EMR such as AllScripts, Cerner, Epic, Athena plus more, all using a pluggable system. Pluggable delivery connectors may be written in any language for custom destinations such as insurance companies and release services. Connectors (2) assist in creating these new plugins. The present disclosure solves this by providing one consistent method and point of contact for the Recipient to specify any validated delivery methods they have available. The IO uses this information to deliver the requested HI as specified by the Recipient using their normal ROI processes.

Further, the present disclosure allows healthcare entities, private practices and hospitals, a simple method of connecting to a multitude of other healthcare providers, private practices, and hospitals, and optionally others such as a patient's PHR or anyone who needs to receive a copy of a patient's medical record securely and confidentially.

Further, the present disclosure provides a plurality of connection methods allowing healthcare providers to connect to the proper recipient's EMR or other desired method of receiving the ROI (fax, email, etc.). To receive anything other than Fax, Email, Print the recipient must sign up for the service and provide connection information which is then validated. Gateway Controller (16), also called Core, may put the information into a common format used by the Delivery Controller (27) and each delivery Connector (17, 18, 19, 20) (referring to FIG. 18) may convert it into the proper format for the Recipient device.

Further, this may provide a consistent common connection point for a healthcare provider to connect to multiple other healthcare providers delivering a patient's record securely and confidentially using the data format and protocol required by each of the other healthcare providers.

Further, the present disclosure describes a method for facilitating delivering of medical information of patients. The method may include a step of receiving, using a communication device, one or more ROIs (Release Of Information) requests for receiving one or more medical information of one or more patients from one or more source devices. Further, the method may include a step of analyzing, using a processing device, the one or more ROIs using one or more dynamic configurations. Further, the method may include a step of identifying, using the processing device, one or more delivery devices based on the analyzing of the one or more ROIs. Further, the method may include a step of determining, using the processing device, one or more delivery criteria based on the analyzing of the one or more ROIs. Further, the method may include a step of generating, using the processing device, a delivery version of the one or more medical information associated with the one or more patients based on the unprocessed version of the one or more medical information and the one or more delivery criteria. Further, the method may include a step of converting, using the processing device, the delivery version of the one or more medical information into a specific device type version of the one or more medical information using one or more dynamic profiles. Further, the method may include a step of transmitting, using the communication device, the specific device type version of the one or more medical information to the one or more delivery devices.

Further, the present disclosure describes a system for facilitating delivering of medical information of patients. The system may include a communication device and a processing device. Further, the communication device may be configured for performing a step of receiving one or more types of ingresses for receiving one or more medical information of one or more patients from one or more source devices. Further, the communication device may be configured for performing a step of transmitting a delivery device specific version of the one or more medical information to the one or more delivery devices. The processing device may be communicatively coupled with the communication device. Further, the processing device may be configured for performing a step of analyzing the one or more types of ingresses using one or more dynamic configurations. Further, the processing device may be configured for performing a step of identifying the one or more delivery devices based on the analyzing of the one or more types of ingresses. Further, the processing device may be configured for performing a step of determining one or more delivery criteria based on the analyzing of the one or more types of ingresses. Further, the processing device may be configured for performing a step of generating the device specific delivery version of the one or more medical information associated with the one or more patients based on the unprocessed version of the one or more medical information and the one or more delivery criteria.

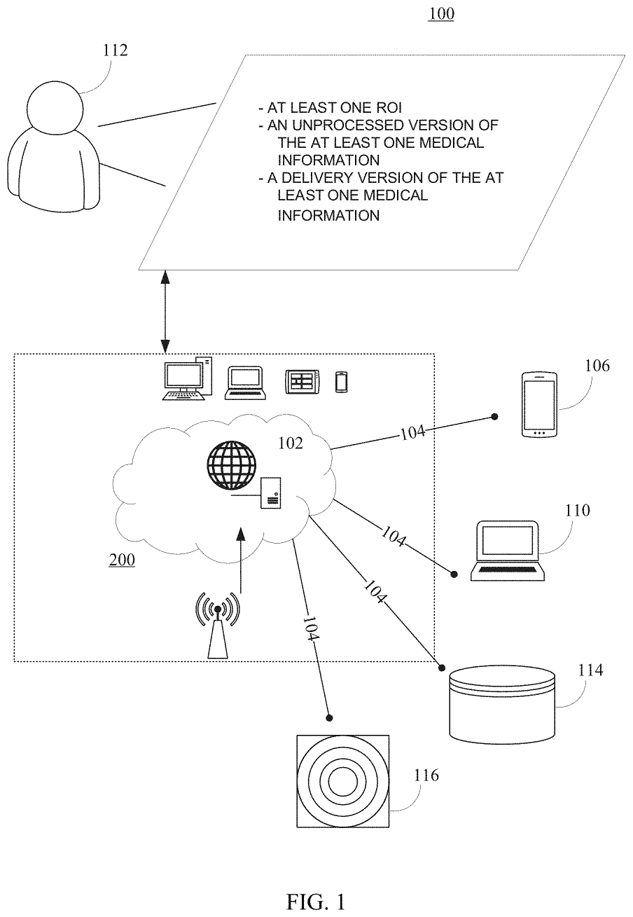

FIG. 1 is an illustration of an online platform 100 consistent with various embodiments of the present disclosure. By way of non-limiting example, the online platform 100 to enable facilitating delivering of medical information of patients may be hosted on a centralized server 102, such as, for example, a cloud computing service in an IO's EMR. The centralized server 102 where the IHIG system may be hosted may communicate with other network entities, such as, for example, a mobile device 106 (such as a smartphone, a laptop, a tablet computer, other servers hosting physician's EMRs, etc.), other electronic devices 110 (such as desktop computers, server computers, etc.), databases 114, and EMR/Electronic repository 116 over a communication network 104, such as, but not limited to, the Internet. Further, users of the online platform 100 may include relevant parties such as, but not limited to, IO based users, Health Information Management (HIM) personnel, Health Information Systems (HIS) personnel, HIS Servers, and so on. Accordingly, in some instances, electronic devices operated by the one or more relevant parties may be in communication with the platform.

A user 112, such as recipients, and HIS personnel, may access online platform 100 through a web-based software application or browser. The web-based software application may be embodied as, for example, but not be limited to, a website, a web application, a desktop application, and a mobile application compatible with a computing device 200.

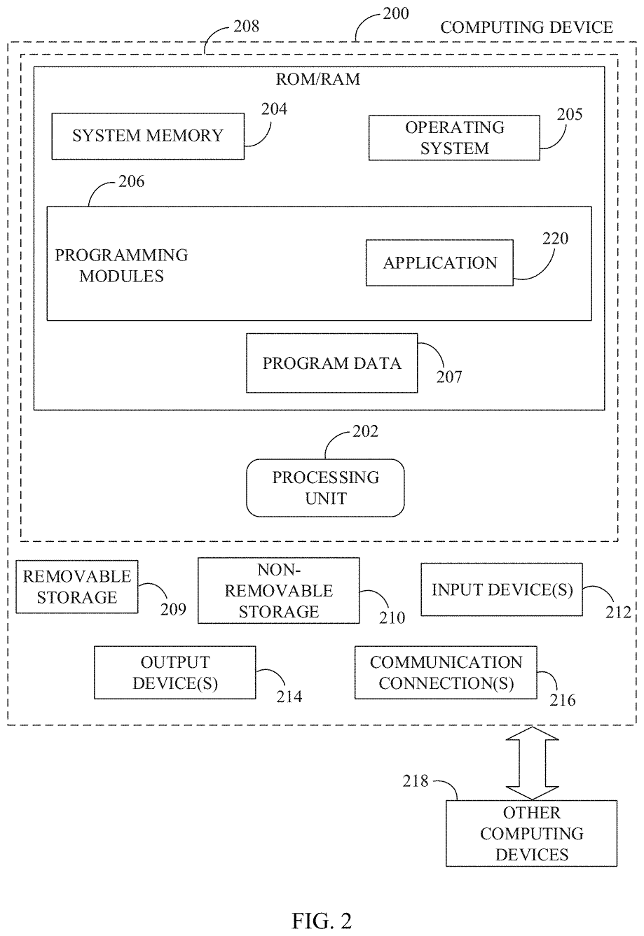

With reference to FIG. 2, a system consistent with an embodiment of the disclosure may include a computing device or cloud service, such as computing device 200. In a basic configuration, computing device 200 may include at least one processing unit 202 and a system memory 204. Depending on the configuration and type of computing device, system memory 204 may comprise, but is not limited to, volatile (e.g., random-access memory (RAM)), non-volatile (e.g., read-only memory (ROM)), flash memory, or any combination. System memory 204 may include operating system 205, one or more programming modules 206, and may include a program data 207. Operating system 205, for example, may be suitable for controlling computing device 200's operation. In one embodiment, programming modules 206 may include image-processing module, machine learning module. In another embodiment, programming modules 206 may include Internal or external Micro services equivalent to another Computing Device (200) for image-processing module, machine learning module and/or image classifying module, and any other process. Furthermore, embodiments of the disclosure may be practiced in conjunction with a graphics library, other operating systems, or any other application program and is not limited to any particular application or system. This basic configuration is illustrated in FIG. 2 by those components within a dashed line 208.

Computing device 200 may have additional features or functionality. For example, computing device 200 may also include additional data storage devices (removable and/or non-removable) such as, for example, magnetic disks, optical disks, or tape. Such additional storage is illustrated in FIG. 2 by a removable storage 209 and a non-removable storage 210. Computer storage media may include volatile and non-volatile, removable and non-removable media implemented in any method or technology for storage of information, such as computer-readable instructions, data structures, program modules, or other data. System memory 204, removable storage 209, and non-removable storage 210 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 200. Any such computer storage media may be part of device 200. Computing device 200 may also have input device(s) 212 such as a keyboard, a mouse, a pen, a sound input device, a touch input device, a location sensor, a camera, a biometric sensor, etc. Output device(s) 214 such as a display, speakers, a printer, etc. may also be included. The aforementioned devices are examples and others may be used.

Computing device 200 may also contain a communication connection 216 that may allow device 200 to communicate with other computing devices 218, such as over a network in a distributed computing environment, for example, an intranet or the Internet. Communication connection 216 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both storage media and communication media.

As stated above, a number of program modules and data files may be stored in system memory 204, including operating system 205. While executing on processing unit 202, programming modules 206 (e.g., application 220 such as a media player) may perform processes including, for example, one or more stages of methods, algorithms, systems, applications, servers, databases as described above. The aforementioned process is an example, and processing unit 202 may perform other processes. Other programming modules that may be used in accordance with embodiments of the present disclosure may include machine learning applications.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, general purpose graphics processor-based systems, multiprocessor systems, microprocessor-based or programmable consumer electronics, application specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

FIGS. 18 through 22 and 26 are descriptive of the IHIGS embodiment being used as a reference within this disclosure.

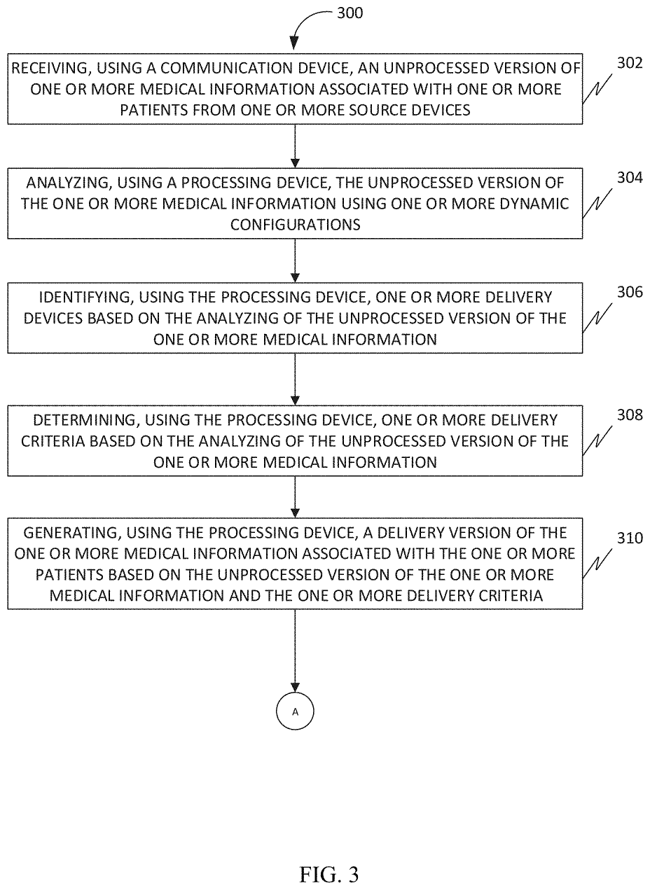

FIG. 3 is a flowchart of a method 300 for facilitating delivering of medical information of patients, in accordance with some embodiments.

Further, the method 300 may include a step 302 of receiving, using the communication device, an unprocessed version of one or more medical information associated with one or more patients from one or more source devices. Further, the unprocessed version of the one or more medical information may include an unprocessed ROI (release of information).

Further, the one or more source devices may be associated with a health entity. Further, the health entity may be an information owner (IO). Further, the one or more medical information may include health information (HI), PHI, PPI, personally identifiable information (PII), medical records, etc. Further, the one or more medical information may include a request of information (ROI), a release of information (ROI). Further, the one or more medical information may include a profile of a recipient. Further, what the profile may include determines how the recipient desires to receive the medical information.

Further, the method 300 may include a step 304 of analyzing, using a processing device, the unprocessed version of the one or more medical information using one or more dynamic configurations. Further, the one or more dynamic configurations are passed around and allow the validation and configuration of various generic processes to a specific purpose for a call. Further, the call may be associated with totally different actions.

Further, the method 300 may include a step 306 of identifying, using the processing device, one or more delivery devices based on the analyzing of the unprocessed version of the one or more medical information. Further, the one or more delivery devices may include a recipient device associated with a recipient. Further, the recipient may include a health entity.

Further, the method 300 may include a step 308 of determining, using the processing device, one or more delivery criteria based on the analyzing of the unprocessed version of the one or more medical information. Further, the one or more delivery criteria may include a document preparation specification, a document format, etc.

Further, the method 300 may include a step 310 of generating, using the processing device, a delivery version of the one or more medical information associated with the one or more patients based on the unprocessed version of the one or more medical information and the one or more delivery criteria. Further, the delivery version of the one or more medical information may include a delivery ROI.

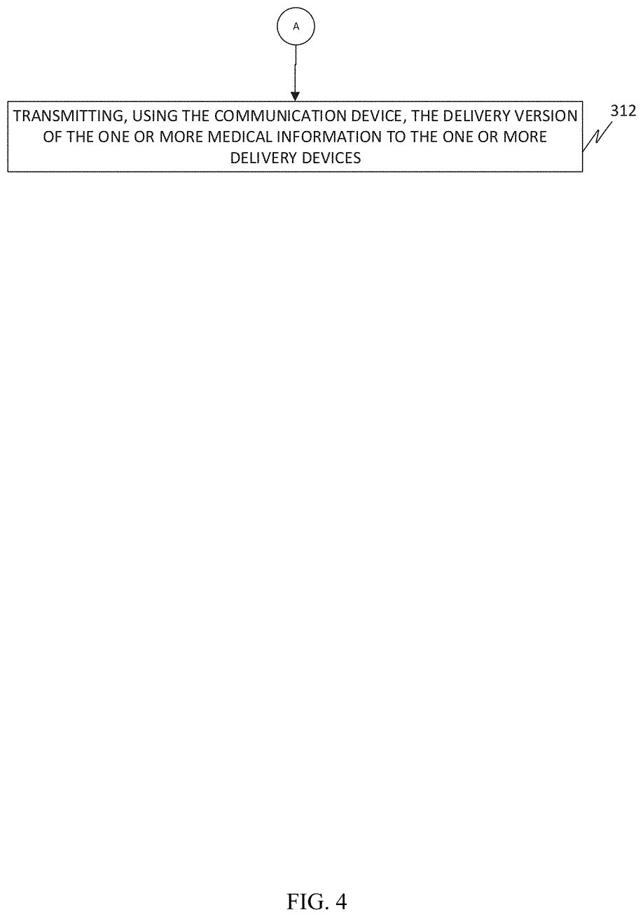

FIG. 4 is a continuation flowchart of FIG. 3.

Further, the method 300 may include a step 312 of transmitting, using the communication device, the delivery version of the one or more medical information to the one or more delivery devices.

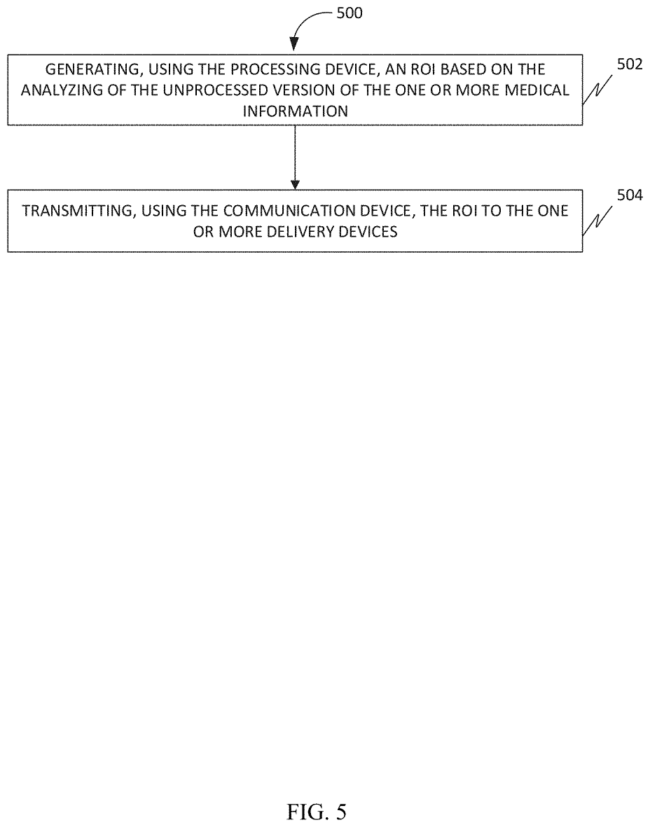

FIG. 5 is a flowchart of a method 500 for facilitating delivering of medical information of patients, in accordance with further embodiments. Further, at 502, the method 500 may include generating, using the processing device, an ROI based on the analyzing of the unprocessed version of the one or more medical information. Further, at 504, the method 500 may include transmitting, using the communication device, the ROI to the one or more delivery devices.

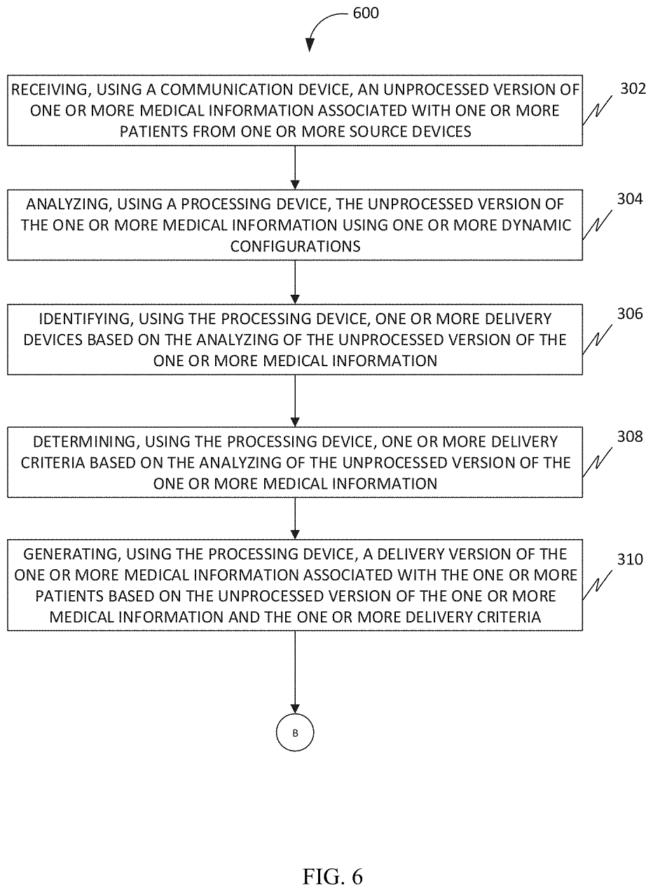

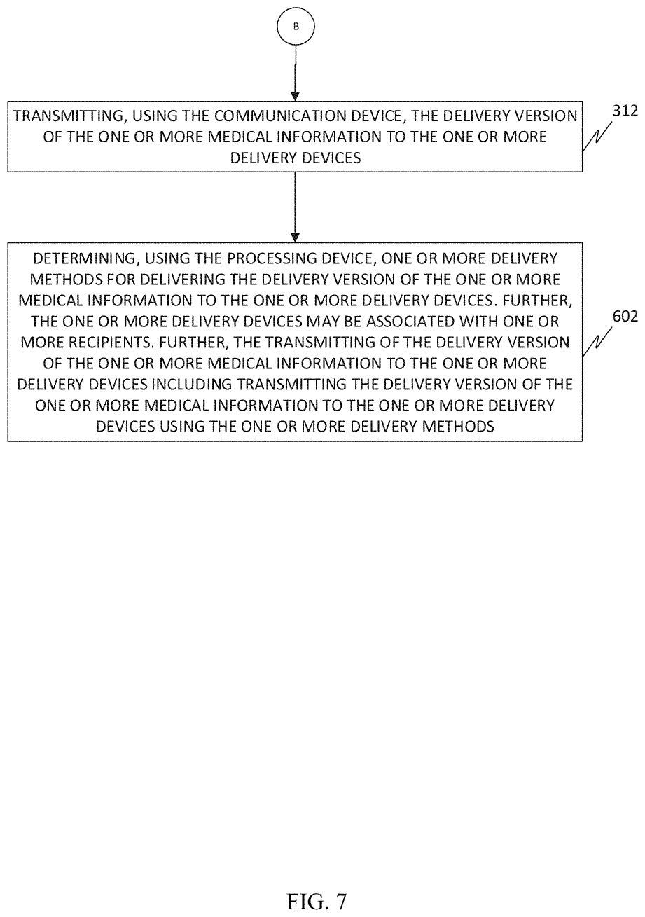

FIG. 6 is a flowchart of a method 600 for facilitating delivering of medical information of patients, in accordance with further embodiments. The steps 302-312 have explained in detail in conjunction with FIG. 3 above. FIG. 7 is a continuation flowchart of FIG. 6. Further, at 602, the method 600 may include determining, using the processing device, one or more delivery methods for delivering the delivery version of the one or more medical information to the one or more delivery devices. Further, the one or more delivery devices may be associated with one or more recipients. Further, the one or more delivery methods may include delivery through email, delivery through fax, delivery through print, delivery through mail, delivery through EMR, etc. Further, the transmitting of the delivery version of the one or more medical information to the one or more delivery devices including transmitting the delivery version of the one or more medical information to the one or more delivery devices using the one or more delivery methods.

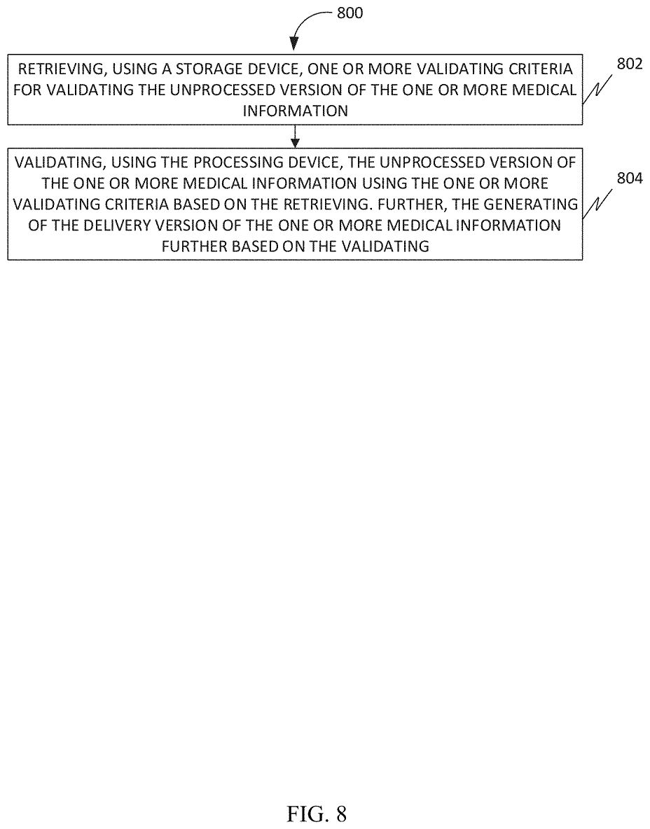

FIG. 8 is a flowchart of a method 800 for facilitating delivering of medical information of patients, in accordance with further embodiments. Further, at 802, the method 800 may include retrieving, using a storage device, one or more validating criteria for validating the unprocessed version of the one or more medical information. Further, the one or more validating criteria may be associated with HIPPA (Health Insurance Portability and Accountability Act). Further, the one or more validating criteria may include HIPPA rules, IO rules, etc. Further, at 804, the method 800 may include validating, using the processing device, the unprocessed version of the one or more medical information using the one or more validating criteria based on the retrieving. Further, the validating may include determining a compliance of the unprocessed version of the one or more medical information. Further, the unprocessed version of the one or more medical information may be validated if the unprocessed version of the one or more medical information complies with the HIPPA rules. Further, the generating of the delivery version (at 310) of the one or more medical information further based on the validating.

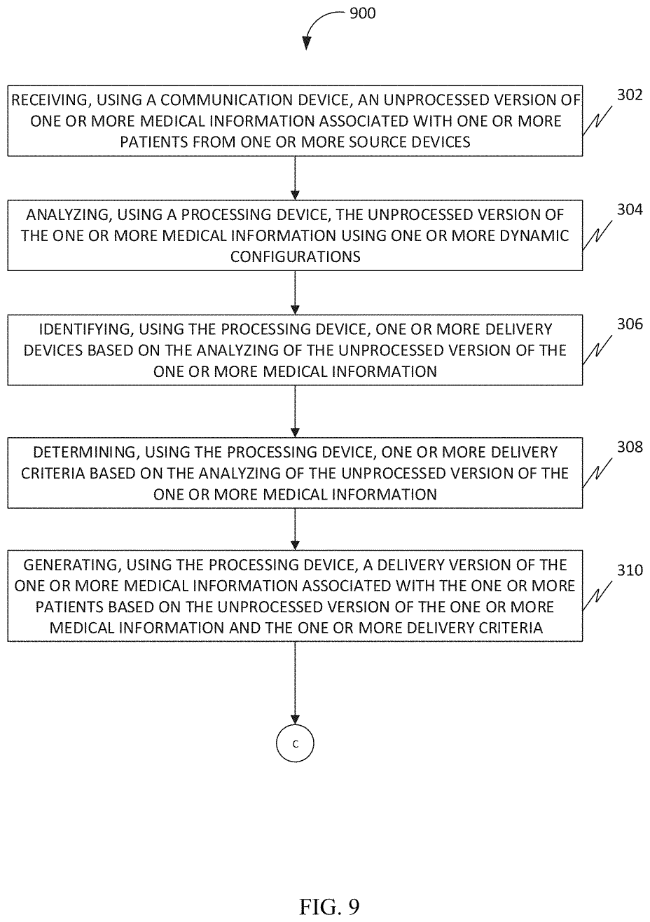

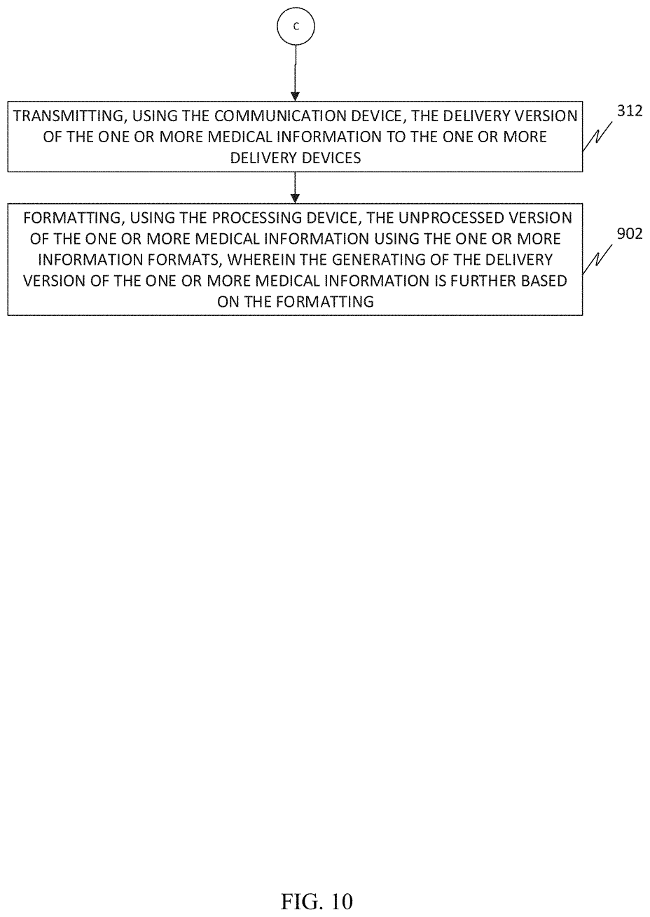

FIG. 9 is a flowchart of a method 900 for facilitating delivering of medical information of patients, in accordance with further embodiments. Further, the one or more delivery criteria may include one or more information formats of the one or more medical information. The steps 302-312 have explained in detail in conjunction with FIG. 3 above. FIG. 10 is a continuation flowchart of FIG. 9. Further, the method 900 may include a step 902 of formatting, using the processing device, the unprocessed version of the one or more medical information using the one or more information formats. Further, the formatting may include converting the unprocessed version into a format of the one or more information formats required by an EMR. Further, the generating of the delivery version (at 310) of the one or more medical information may be further based on the formatting.

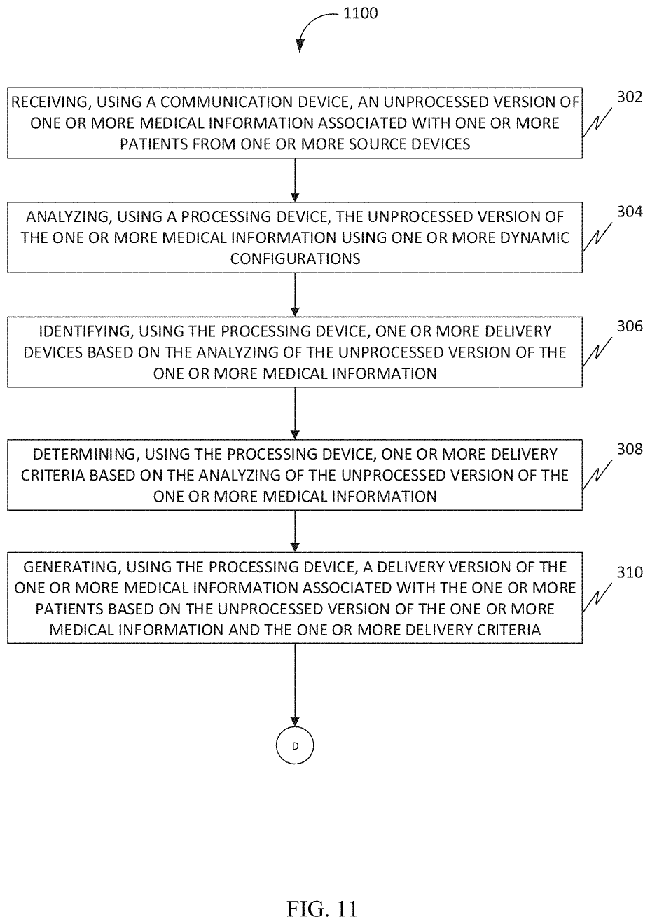

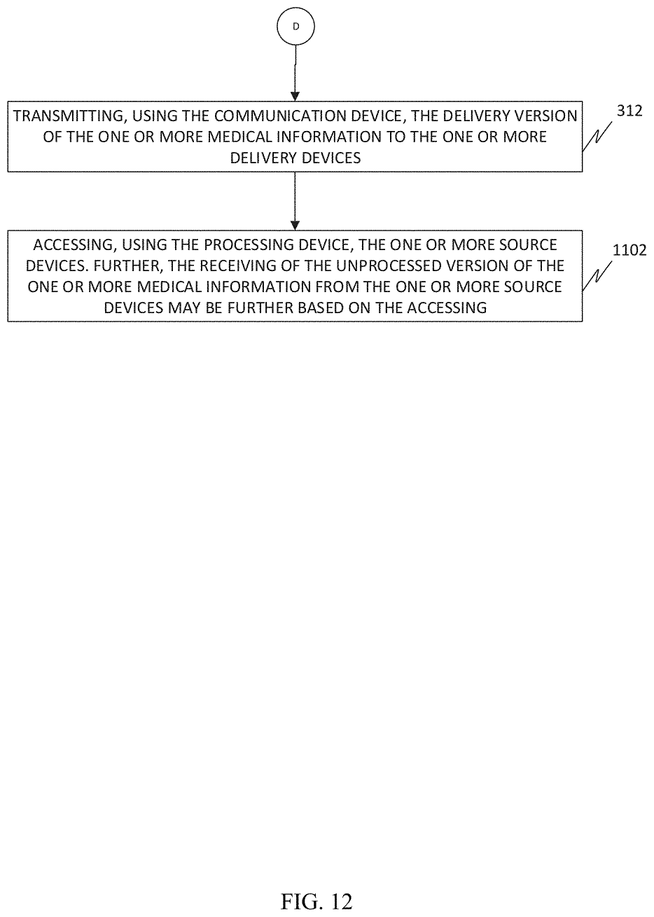

FIG. 11 is a flowchart of a method 1100 for facilitating delivering of medical information of patients, in accordance with further embodiments. The steps 302-312 have explained in detail in conjunction with FIG. 3 above. FIG. 12 is a continuation flowchart of FIG. 11. Further, at 1102, the method 1100 may include accessing, using the processing device, the one or more source devices. Further, the one or more source devices may include EMR devices. Further, the one or more source devices may include the unprocessed version of the one or more medical information of the one or more patients. Further, the receiving of the unprocessed version of the one or more medical information from the one or more source devices may be further based on the accessing.

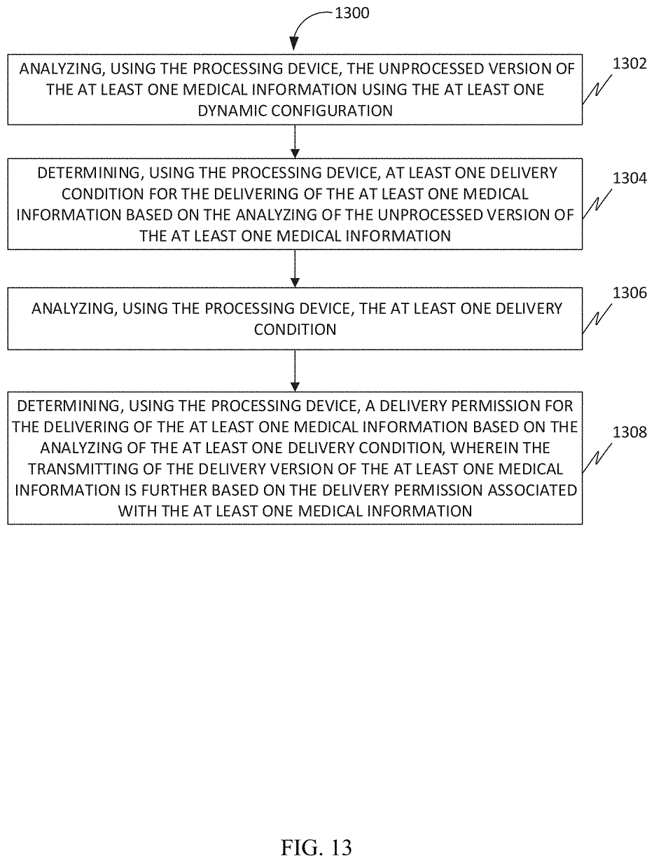

FIG. 13 is a flowchart of a method 1300 for facilitating delivering of medical information of patients, in accordance with further embodiments.

Further, at 1302, the method 1300 may include analyzing, using the processing device, the unprocessed version of the one or more medical information using one or more dynamic configurations.

Further, at 1304, the method 1300 may include determining, using the processing device, one or more delivery conditions for the delivering of the one or more medical information based on the analyzing of the unprocessed version of the one or more medical information. Further, the one or more delivery conditions may include conditions associated with the delivering of the one or more medical information. Further, the conditions may include delivery allowable, delivery restricted, delivery conditional, etc.

Further, at 1306, the method 1300 may include analyzing, using the processing device, the one or more delivery conditions.

Further, at 1308, the method 1300 may include determining, using the processing device, a delivery permission for the delivering of the one or more medical information based on the analyzing of the one or more delivery conditions. Further, the transmitting of the delivery version of the one or more medical information may be further based on the delivery permission associated with the one or more medical information.

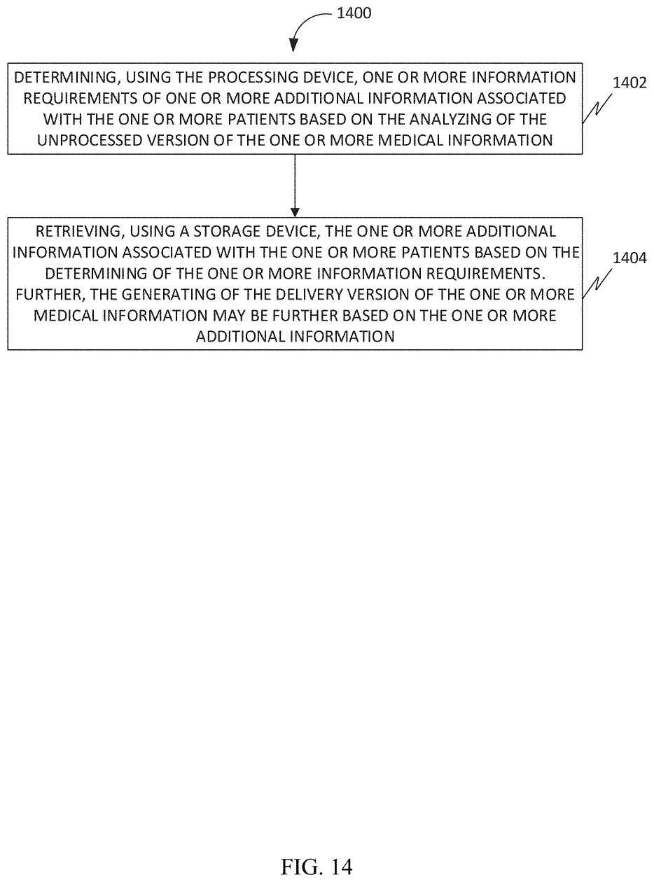

FIG. 14 is a flowchart of a method 1400 for facilitating delivering of medical information of patients, in accordance with further embodiments.

Further, at 1402, the method 1400 may include determining, using the processing device, one or more information requirements of one or more additional information associated with the one or more patients based on the analyzing of the unprocessed version of the one or more medical information. Further, the one or more information requirements may include one or more identifiers of the one or more additional information required by the recipient. Further, the one or more information requirements may include a category, a type, etc. of the one or more additional information required by the recipient.

Further, at 1404, the method 1400 may include retrieving, using a storage device, the one or more additional information associated with the one or more patients based on the determining of the one or more information requirements. Further, the generating of the delivery version (at 310) of the one or more medical information may be further based on the one or more additional information.

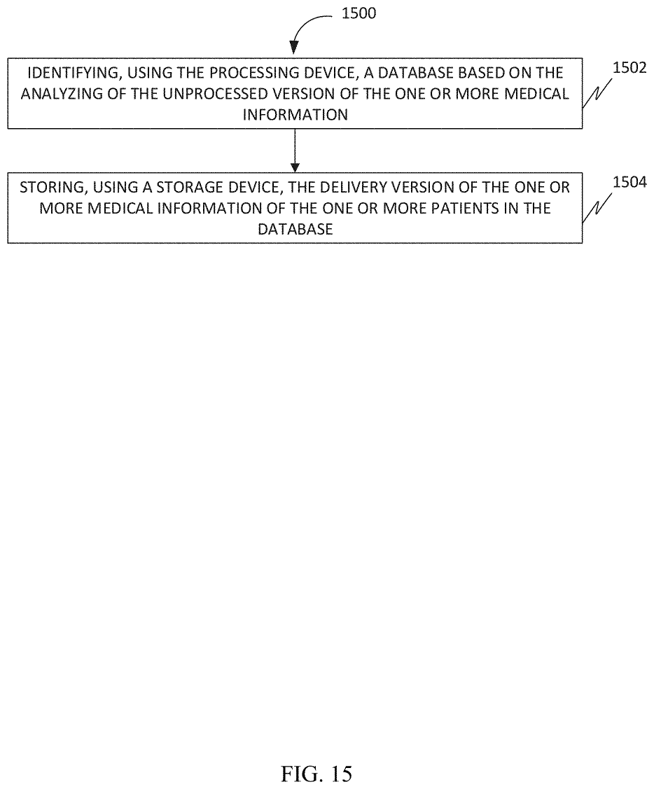

FIG. 15 is a flowchart of a method 1500 for facilitating delivering of medical information of patients, in accordance with further embodiments.

Further, at 1502, the method 1500 may include identifying, using the processing device, a database based on the analyzing of the unprocessed version of the one or more medical information.

Further, at 1504, the method 1500 may include storing, using a storage device, the delivery version of the one or more medical information of the one or more patients in the database.

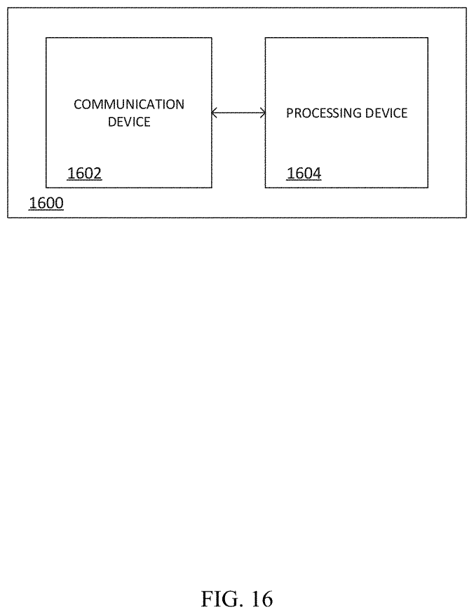

FIG. 16 is a block diagram of a system 1600 for facilitating delivering of medical information of patients, in accordance with some embodiments. The system 1600 may include a communication device 1602 and a processing device 1604.

Further, the communication device 1602 may be configured for performing a step of receiving an unprocessed version of one or more medical information associated with one or more patients from one or more source devices. Further, the one or more source devices may be associated with a health entity. Further, the health entity may be an information owner (IO). Further, the one or more medical information may include health information (HI), PHI, PPI, personally identifiable information (PII), medical records, etc. Further, the one or more medical information may include a request of information (ROI), a release of information (ROI). Further, the one or more medical information may include a profile of a recipient. Further, what the profile may include determines how the recipient desires to receive the medical information.

Further, the communication device 1602 may be configured for performing a step of transmitting a delivery version of the one or more medical information to one or more delivery devices.

The processing device 1604 may be communicatively coupled with the communication device 1602.

Further, the processing device 1604 may be configured for performing a step of analyzing the unprocessed version of the one or more medical information using one or more dynamic configurations.

Further, the processing device 1604 may be configured for performing a step of identifying the one or more delivery devices based on the analyzing of the unprocessed version of the one or more medical information. Further, the one or more delivery devices may include a recipient device associated with a recipient. Further, the recipient may include a health entity.

Further, the processing device 1604 may be configured for performing a step of determining one or more delivery criteria based on the analyzing of the unprocessed version of the one or more medical information. Further, the one or more delivery criteria may include a document preparation specification, a document format, etc.

Further, the processing device 1604 may be configured for performing a step of generating the delivery version of the one or more medical information associated with the one or more patients based on the unprocessed version of the one or more medical information and the one or more delivery criteria.

In some embodiments, the processing device 1604 may be configured for performing a step of generating an ROI based on the analyzing of the unprocessed version of the one or more medical information. Further, the communication device 1602 may be configured for performing a step of transmitting the ROI to the one or more delivery devices.

In some embodiments, the processing device 1604 may be configured for performing a step of determining one or more delivery methods for delivering the delivery version of the one or more medical information to the one or more delivery devices. Further, the one or more delivery devices may be associated with one or more recipients. Further, the one or more delivery methods may include delivery through email, delivery through fax, delivery through print, delivery through mail, delivery through EMR, etc. Further, the transmitting of the delivery version of the one or more medical information to the one or more delivery devices including transmitting the delivery version of the one or more medical information to the one or more delivery devices using the one or more delivery methods.

In some embodiments, the one or more delivery criteria may include one or more information formats of the one or more medical information. Further, the processing device 1604 may be configured for performing a step of formatting the unprocessed version of the one or more medical information using the one or more information formats. Further, the generating of the delivery version of the one or more medical information may be further based on the formatting.

In some embodiments, the processing device 1604 may be configured for performing a step of accessing the one or more source devices. Further, the one or more source devices may include the unprocessed version of the one or more medical information of the one or more patients. Further, the receiving of the unprocessed version of the one or more medical information from the one or more source devices may be further based on the accessing.

In some embodiments, the processing device 1604 may be configured for performing a step of analyzing the unprocessed version of the one or more medical information using the one or more dynamic configurations. Further, the processing device 1604 may be configured for performing a step of determining one or more delivery conditions for the delivering of the one or more medical information based on the analyzing of the unprocessed version of the one or more medical information. Further, the processing device 1604 may be configured for performing a step of analyzing the one or more delivery conditions. Further, the processing device 1604 may be configured for performing a step of determining a delivery permission for the delivering of the one or more medical information based on the analyzing of the one or more delivery conditions. Further, the transmitting of the delivery version of the one or more medical information may be further based on the delivery permission associated with the one or more medical information.

In some embodiments, the communication device 1602 may be configured for performing a step of receiving one or more patient data of the one or more patients from one or more patient monitors. Further, the one or more patient monitors may include one or more biological sensors. Further, the one or more biological sensors may be configured for generating the one or more patient data based on detecting one or more biological metrics of the one or more patients. Further, the processing device 1604 may be configured for performing a step of analyzing the one or more patient data. Further, the processing device 1604 may be configured for performing a step of determining a health condition of the one or more patients based on the analyzing of the one or more patient data. Further, the determining of the one or more information requirements is further based on the determining of the health condition.

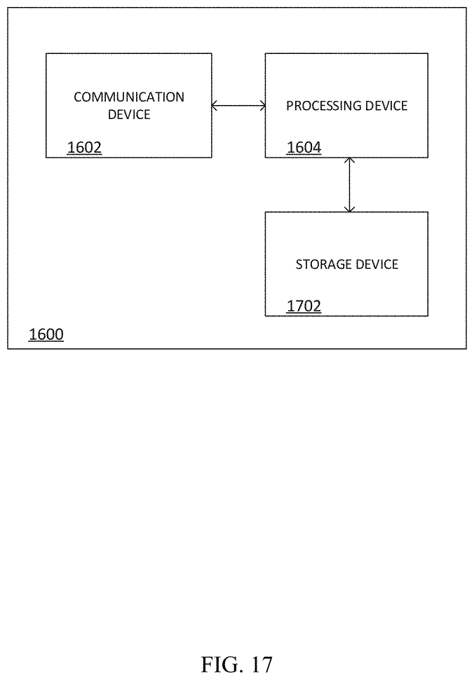

FIG. 17 is a block diagram of the system 1600 for facilitating delivering of medical information of patients, in accordance with some embodiments. The system 1600 may further include a storage device 1702.

Further, the storage device 1702 may be communicatively coupled with the processing device 1604. Further, the storage device 1702 may be configured for retrieving one or more validating criteria for validating the unprocessed version of the one or more medical information. Further, the one or more validating criteria may be associated with HIPPA (Health Insurance Portability and Accountability Act). Further, the one or more validating criteria may include HIPPA rules, IO rules, etc. Further, the processing device 1604 may be configured for performing a step of validating the unprocessed version of the one or more medical information using the one or more validating criteria based on the retrieving. Further, the validating may include determining a compliance of the unprocessed version of the one or more medical information. Further, the unprocessed version of the one or more medical information may be validated if the unprocessed version of the one or more medical information complies with the HIPPA rules. Further, the generating of the delivery version of the one or more medical information further based on the validating.

In some embodiments, the processing device 1604 may be configured for performing a step of identifying a database based on the analyzing of the unprocessed version of the one or more medical information. Further, the storage device 1702 may be configured for storing, using a storage device, the delivery version of the one or more medical information of the one or more patients in the database.

In some embodiments, the processing device 1604 may be configured for performing a step of determining one or more information requirements of one or more additional information associated with the one or more patients based on the analyzing of the unprocessed version of the one or more medical information. Further, the one or more information requirements may include one or more identifiers of the one or more additional information required by the recipient. Further, the one or more information requirements may include a category, a type, etc. of the one or more additional information required by the recipient. Further, the storage device 1702 may be configured for performing a step of retrieving the one or more additional information associated with the one or more patients based on the determining of the one or more information requirements. Further, the generating of the delivery version of the one or more medical information may be further based on the one or more additional information.

FIG. 18 is a block diagram of a system 1800 for facilitating the exchange of data, especially medical data associated with a patient, in accordance with some embodiments of the present disclosure. Accordingly, the present disclosure may allow any IO to connect to a multitude of other EMR systems and deliver a patient's health record securely and confidentially directly into a Recipient's EMR. For example, a Recipient in one case requesting HI from an IO may now be the IO providing HI to a Recipient that was the IO in the original case. In other words, the present disclosure may be set up to be a two-way street.

Further, the present disclosure may include a plurality of connection modules allowing IOs to interface an apparatus that allows connection to the Recipient's EMR or other devices the Recipient may have capable of receiving the Release of Information requests (ROI) such as fax, email, etc. To receive anything other than fax, email, print, needs to provide connection and credentials information for their EMR. Further, the present disclosure may include Gateway Controller (16) that may put the information into a common format and each delivery module (17, 18, 19, 20) converts the HI into a proper format for the Recipient's specific device type.

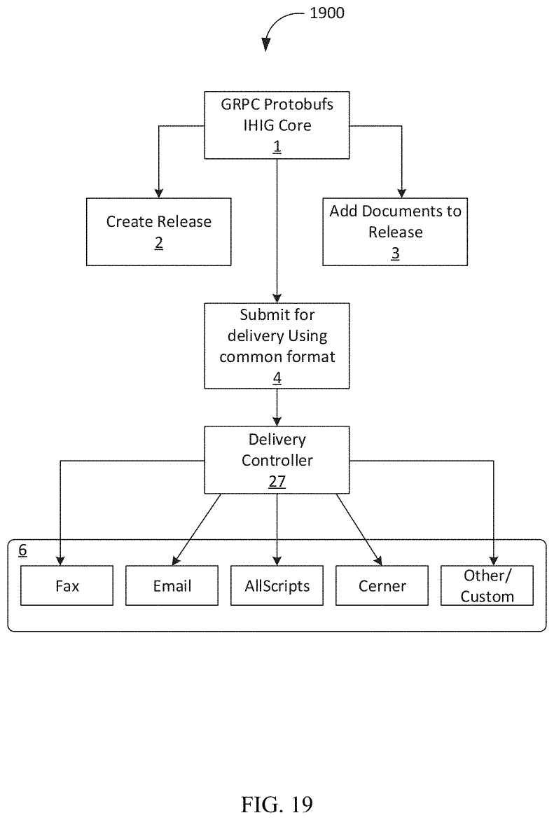

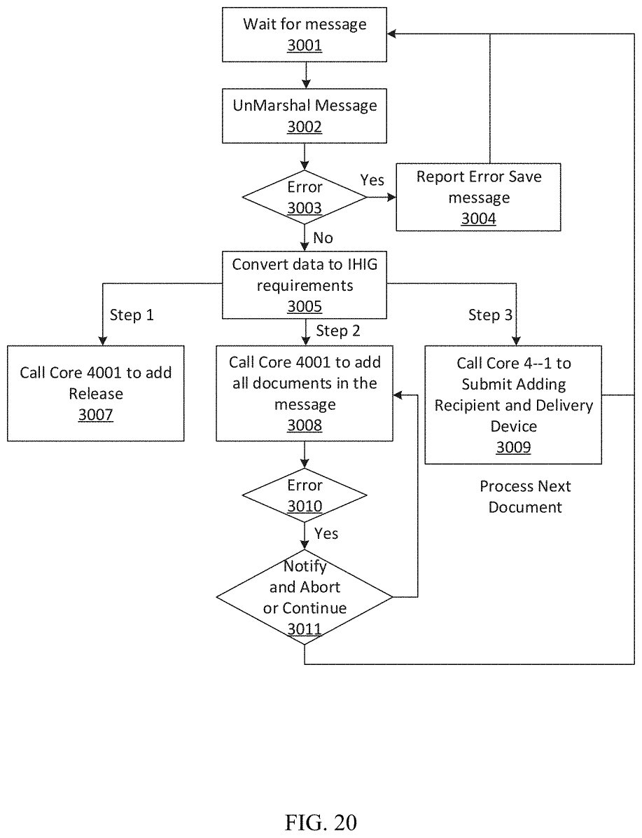

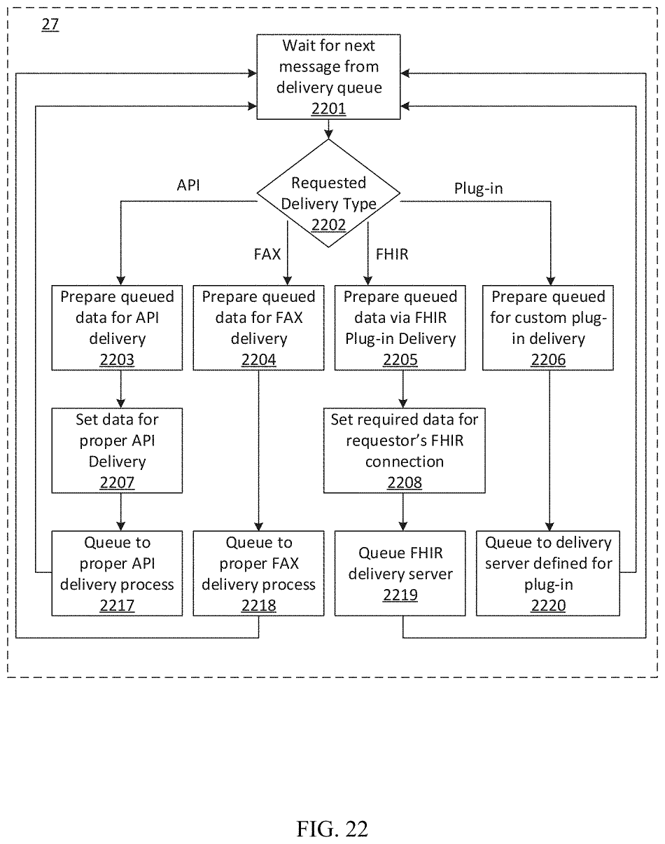

Further, the present disclosure may allow a multitude of healthcare providers (IOs) (71, 72, 73, 74), each providing care to a multitude of patients. Further, each of the healthcare providers may deliver the HI to a particular external physician, other hospital, insurance company, legal office, government organization, patient, or any other entity that requests and signs a valid release request (ROI), and one or more supported delivery method (17, 18, 19, 20) (Recipients). Each Recipient may be required to register their delivery destinations, methods, and security on an admin GUI (26). Further, each healthcare provider (IO) desiring to delivery PPI/PHI may connect to a (IO) using input methods (8) based on a choice of each healthcare provider (IO). The present disclosure may be flexible and allow additional input method servers (12, 13) as required by a healthcare provider (IO) and provided by the implementer of the present patent. The ingress processes accept a message based on the type on ingress and convert it into a common layout and delivering it to the Gateway Controller (16). Further, a new input method provided by a secure plug-in service may be created and installed to accept the information as presented by the healthcare provider, prepare it into the format required by a Gateway Controller (16), and delivering it to the gateway. Further, one thread of the OS of a system running the receive using ingress only accepts on release for one patient and multiple delivery devices at a time. Further, input servers may use the GRPC or Restful Ingress methods directly using the proper format without any additional interfaces. The message Queue Ingress method as shown in FIG. 20, or any new pluggable Ingress method easily created, using a Connector or other methods are available for the IO directly using the proper format without any additional interfaces. The Gateway Controller (16) accepts the PHI/PII being released, patient's, and IO's details, may determine the proper authorizations to directly access HI on the IO, extra details including but not limited to patient details, healthcare provider details, destination details, and delivery details that must be requested and used in logging the delivery of PHI. Further, the Gateway Controller shown in FIG. 19, may determine the proper message type received and use the proper connector for further processing. Further, the Gateway Controller 16, as shown in FIG. 18, may determine the proper delivery destinations to be used. Further, the Gateway Controller (16) may provide the information to the Delivery Controller (27). Delivery Controller (27), as shown in FIG. 22, may prepare the delivery by creating a common delivery message used by the supported methods (17, 18, 19, 20) which may further modify the data into that required by the actual delivery device. Further, the API Delivery Method (17) may include Allscripts TouchWorks EMR, any other EMR, or electronic repository. Further, the information from the healthcare provider (IO) and the profile established for the Recipient (15) in the gateway may be used together to save the PHI in the patient's record in the recipient's Allscripts TouchWorks EMR. Further, the healthcare provider (IO) may send the requested HI to a method to deliver it electronically to the recipient on any registered EMR or other types of delivery modules. Further, limited patient information, including some personally identifiable information (PII) may be encrypted and stored in a database (15) that may be associated with the release for an implementer's specified time post-delivery to allow for easy redelivery, auditing, or any other purpose. Further, the system may store the medical records received in each submission and maintain them after the medical records have been delivered to the proper destinations based upon the implementer's specification. The electronic (EMR, or other devices that electronically place the PHI in an Electronic Repository) recipients manage their own profiles using the Administration Graphic user Interface (26). They are solely responsible for the correctness, though the present disclosure validates the provided information. Further, the system may include a multitude of delivery methods (17, 18, 19, 20) and Ingestion Methods (8). Further, the system may include a new delivery method, such as Plugin Delivery Method (20) using a Connector FIG. 26. for receiving the common dataset and configurations from the Delivery Controller (27), that a particular recipient requires. Further, the present disclosure may include additional ingestion methods that may provide the Gateway Controllers (16) input requirements from the IO's format.

FIG. 19 is a flow diagram of a general delivery method 1900 using a GRPC protocol with buffers, and Restful standards in accordance with some embodiments. FIG. 26 is a flow diagram related to the technology used by the Connector to make the creation of new services easier and faster. Connectors allow both dynamic startup configuration and per message configuration. For example, an Email Delivery Connector can be created that can interface with two different email servers, one for regular in-house insecure messages and one for high security external messages. The startup tells the connector what it is, an Email Delivery Service, and how the delivery message will be supplied, via Restful HTTP. The connector could be built to accept any protocol. This sets up the Connector to be able to receive messages telling it how to deliver the HI in the current message. When the delivery message is received. It contains the real time configuration for the secure delivery containing the URL, the security requirements to access the server, and anything else that is needed to deliver the HI. The messages also contain the necessary information of the recipient required for delivery. As another example, an EMR vendor has a current version of their API and has just released a Beta test version and the present disclosure has been installed for some time with a connector for the current version of the EMR's API. To support this new version, the implementer would need to create a new version of the Vendor's EMR Connector. In this Connector, there are two changes that must be made to support both versions. Code to support the new API and a version check to call either the original or BETA version based upon the current configuration provided at runtime. Once this new Connector is deployed, any Recipient in the Beta program for the EMR can automatically participate by changing their EMR version in their profile via the Admin Console (1-26). If they want to switch out of the Beta just change their profile back. When the Beta is formally released, the only change for all users is to change the default configuration passed in the delivery message, plus the Beta participants should change their active version to the new one or blank.

FIG. 20 is a flow diagram related to a basic implementation of a message queue ingress, in accordance with some embodiments. Accordingly, the message queue ingress may include a plugin that uses a message broker such as RabbitMQ™ to deliver the ROI from the IO to the present disclosure in the present disclosure. Further, the present disclosure may include a default message queue Ingress where the health provider writes the message following the present disclosure standard message format. This example is a Message Queue translator that accepts a message from ChartArchive, the example medical record archive system, that currently provides an ROI delivery message to their fax and print service. This would require a connector to accept the ChartArchive message protocol, RabbitMQ™, and process it into the present disclosure. Further, at 3001. this service is always waiting for a new message to arrive and receives the message upon arrival. Further, the message may be in JSON, or another format defined by the IO and is UnMarshaled (3002). Further, the ROI may include a multitude of Documents to be delivered. Errors are checked (3003) and if an error exists, the error information is logged, the message is saved, the IO may be notified, and the current process is aborted returning to the wait for a message (3001).

STEP 1. If there is no error, the release data is extracted (3005) and converted to the internal format to submit through the Gateway Controller 16 (referring to FIG. 18). Once the information is extracted and the Release is created using Release Service 5 (referring to FIG. 18).

STEP 2. Each document in the ROI is extracted and converted to the IHIGS's internal format. In this example, this conversion process may include contacting the ChartArchive server via its connector FIG. 26. to get additional information. When the Document is valid, the Gateway Controller 16 (referring to FIG. 18) is used to add the new document URL to the Release. If the incoming message had the actual image, the Gateway Controller may add the image to the internal document immediately. If the message contains a URL indicating where to request the image, instead of the actual image, the Gateway Controller may store the URL and may use the Connector to immediately extract the image. Another simple option is for the Gateway Controller to postpone extracting and storing the image until it is needed during delivery. In both cases, the Gateway Controller may be called to use the Connector to retrieve the image when needed. The delivery process is the same in either case. After all the documents have been added to the release, the recipient and their delivery device are extracted.

STEP 3. This information may be combined with the Release Information creating a Delivery Request and sent to the Delivery Controller 27 (referring to FIG. 18). Further, the core may let the Delivery Controller 2) know there is new work to do. This process is complete and Wait for Message (3001) takes over.

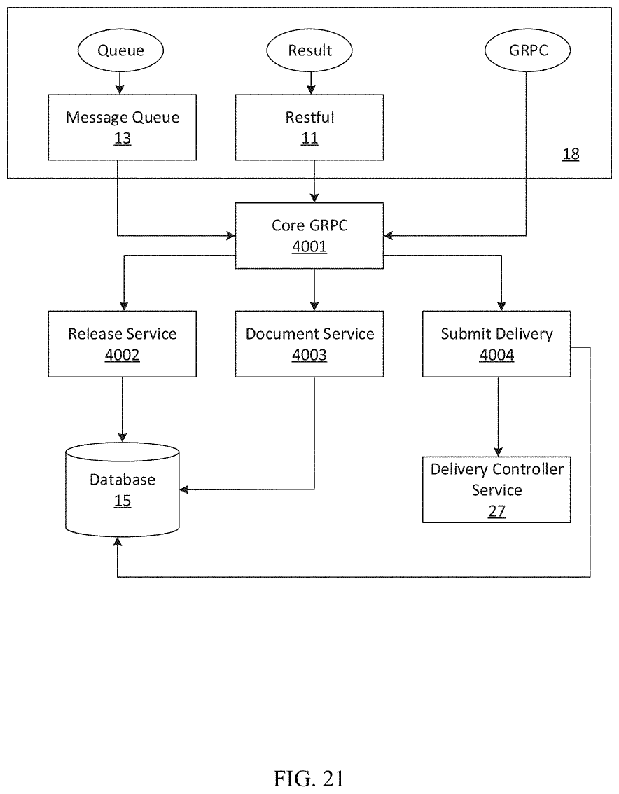

FIG. 21 is a flow diagram associated with a Gateway Controller (Core) as a GRPC protocol and buffers or Restful, in accordance with some embodiment. The present disclosure supports total functionality and capability definition via real time dynamic configurations using Connectors (2). For example, new message types to provide additional functionality sent to the Gateway Controller are easily configured using Connectors (2).

Accordingly related to ROI delivery, the default message type, the IO may submit the ROI to the Gateway using GRPC and Protocol Buffers. Further, the additional ingress methods (or modules) may include HTTP, Message Queues, Command-Line, and so on. Depending on the Ingestion method (11, 12, 13) (referring to FIG. 18) that may be selected, the method may include accessing the IO to retrieve any additional required information. Further, the entry point to the GRPC/Restful (11) service may include a Core that manages various subsystems and manages all security. Normally, everything that needs to be done to process a new release is done through the Core using Connectors and other services, though the implementer may use the other services and Connectors used by Core directly as needed. Others like Message queues may provide all the information in the one package they deliver to the ingress. Images of each document may be provided directly in the message, as a URL, or other references to request the document image directly from the IO's internal systems. Further, a document is an inclusive description that includes PDF, TIFF, FHIR, and others.

FIG. 22 is a flow diagram associated with the delivery controller, in accordance with some embodiments.

STEP 1—A notification from the Gateway Controller (16) starts the delivery process for the Release and creates a standard message to be queued to the proper Deliver Process. Further, the Delivery Controller may use a standard message format to communicate with each Delivery Service. A message is received providing a reference to the information of a Release that must be delivered.

STEP 2—The Delivery Controller (2202) determines the device to be used to deliver the Release and uses the proper delivery proper connector for the specified delivery device.

STEP 3—Contacts Core requesting the current version of the Release. This may be returned without the actual image. If the image has already been received, requested, or extracted by the present disclosure the image reference returned is the URL to the internal copy of the image, otherwise, it is the URL of the external Image at the IO. Any additional information required for delivery such as Document Class/Type, Title . . . is requested using the Connector (26) for that IO. At this point, the Release has everything it needs to be delivered to the Recipient except the actual images.

STEP 4—This step may be used with connectors that require additional processing to collect all the required information for delivery. Normally using an API or FHIR delivery device will require some additional processing items (2207 and 2208). Custom delivery devices may require this step also. This updated message is queued to the actual Delivery Device Connector (2207) The Connector selects each document in the Release individually and requests the image for that document from Core. Core passes the request on to the Image Manager service which uses the Image URL and requests it from the proper Connector (2). This may be one for the IO or an internal repository. This image is then returned to the Delivery Device Connector.

STEP 5—At this point, the release is completely ready to be delivered. Delivery is started by calling the proper Connector (2217, 2218, 2219 2220). Each Delivery Device Connector knows how to connect to its EMR brand and model and performs the actual insertion of the patient record as required by that API. If successful, a success message is sent to the Delivery Controller 27 (referring to FIG. 18). For example, an AllScripts Connector for TouchWorks will work with all Recipients who use the TouchWorks EMR combining the Connectors knowledge on how to add the Document to the EMR in the proper patient's account and under the proper document type along with the proper configuration information and special instructions are provided to the Connector in the delivery message.

For example, a delivery to AllScripts uses an API to communicate with the AllScripts server to accept information for the Recipient. A Connector is created to handle AllScripts deliveries and knows how to convert the Delivery Controller message format to all the various methods the AllScripts API uses to process the new information being added to a patient's chart. This Connector would be a type 1-17.

Further, the Delivery Controller may use a standard message format to communicate with each Delivery Service. Further, in the queue ingress, as shown in FIG. 20, and referencing the ChartArchive example may include delivery services for any possible service supported by the present disclosure. The delivery service may not care because the ChartArchive environment and configuration specification specifies an external converter service (2207, 2208) and the proper Conversion Connector to use to convert, which does the conversion, and sends the converted message on to the actual Delivery Device Connector. This multi-connector approach makes it easier to create delivery device services. It could also be done in other ways such as one package that does all that is required and communicates within itself. Further, the external converter receives the response from the delivery service and converts the response into the standard present disclosure response which is delivered back to the Delivery Controller. Delivery by additional services may use the preferred message format, however, as shown above, it may be very flexible to work with any delivery service.

STEP 6—The Delivery Controller message queue (2201) receives the response messageback from the Connector and if successful notifies the Release Service. If it failed, depending on the reason, for example, the fax line is busy, it may either be re-queued, or both the Recipient and the IO notified of the error. In another example, if the error is a missing or invalid Medical Record Number, the Recipient may be notified to go into the Admin GUI (26), correct the issue, and submit it again.

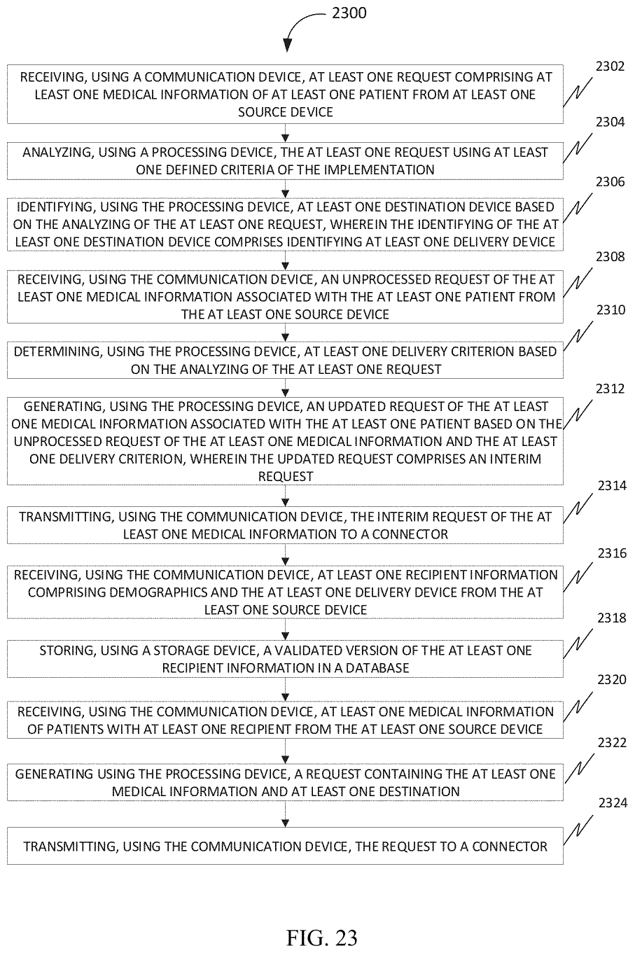

FIG. 23 is a flowchart of a method 2300 for facilitating delivering of medical information of patients to a requestor's device, in accordance with some embodiments. Accordingly, the method 2300 may include a step 2302 of receiving, using a communication device, at least one request comprising at least one medical information of at least one patient from at least one source device. Further, the method 2300 may include a step 2304 of analyzing, using a processing device, the at least one request using at least one defined criteria of the implementation. Further, the method 2300 may include a step 2306 of identifying, using the processing device, at least one destination device based on the analyzing of the at least one request, wherein the identifying of the at least one destination device comprises identifying at least one delivery device. Further, the method 2300 may include a step 2308 of receiving, using the communication device, an unprocessed request of the at least one medical information associated with the at least one patient from the at least one source device. Further, the method 2300 may include a step 2310 of determining, using the processing device, at least one delivery criterion based on the analyzing of the at least one request. Further, the method 2300 may include a step 2312 of generating, using the processing device, an updated request of the at least one medical information associated with the at least one patient based on the unprocessed request of the at least one medical information and the at least one delivery criterion, wherein the updated request comprises an interim request. Further, the method 2300 may include a step 2314 of transmitting, using the communication device, the interim request of the at least one medical information to a connector. Further, the method 2300 may include a step 2316 of receiving, using the communication device, at least one recipient information comprising demographics and the at least one delivery device from the at least one source device. Further, the method 2300 may include a step 2318 of storing, using a storage device, a validated version of the at least one recipient information in a database. Further, the method 2300 may include a step 2320 of receiving, using the communication device, at least one medical information of patients with at least one recipient from the at least one source device. Further, the method 2300 may include a step 2322 of generating using the processing device, a request containing the at least one medical information and at least one destination. Further, the method 2300 may include a step 2324 of transmitting, using the communication device, the request to a connector. Further, one or more medical information is received. Further, one or more steps of the method 2300 may form a loop such as receiving the records from the source, and processing of each medical information using one or more steps of the method 2300. Also, there can be only one patient in a medical information. Each received medical information must have at least one delivery device. Further, the method 2300 is capable of handling more than one of these requests at a time, however, each is one ROI. Further, the ROI is the medical information. Further, the ROI is a specific request of a IO for at least one medical report of one patient that is delivered to at least one delivery device. Each receive is from one source at a time. Many receives can go on at the same time. The unprocessed version of the medical information does not contain delivery information, instead, it contains a list of physicians which are looked up in a configuration database FIG. 18-26 by the physician identifier and each document received. If the physician wants that type of document automatically delivered, a new normal release is created using the one document and the physician's delivery devices return from the lookup. After that no difference. For example. A face sheet (billing information) when one is created in the hospital their system automatically sends it and all of the physicians associated with the patient. In a normal situation, all consulting physicians want that information, however, the primary care physician does not since they already have it.

In further embodiments, the method 2300 may include a step of determining, using the processing device, whether required information for delivery is comprised in the unprocessed request. Further, the method 2300 may include a step of extracting, using the storage device, various segments from the unprocessed request, wherein the various segments are stored. Further, the method 2300 may include a step of analyzing, using the processing device, the segments comprised in the unprocessed request.

In further embodiments, the method 2300 may include a step of analyzing, using the processing device, the extracted segments using at least one dynamic configuration.

Further, in some embodiment, the identifying of the at least one delivery device comprises validating that the at least one delivery device is proper. Further, the identifying of the at least one delivery device comprises validating that the at least one delivery device is active, wherein the method 2300 further comprises storing, using the storage device, a status of the at least one delivery device.

Further, in some embodiment, the determining of the at least one delivery criterion comprises validating whether an electronic medical record system is configured properly. Further, the determining of the at least one delivery criterion comprises validating other delivery devices.

Further, in some embodiment, the generating of the updated request comprises creating the updated request with confirmed sub elements, wherein the confirmed sub elements comprise one or more of the at least one delivery device, the at least one recipient, and at least one document to be delivered. Further, the generating of the updated request comprises transmitting the update request to a delivery connector for a specified delivery device type. Further, the generating of the updated request comprises receiving a delivery status from the delivery connector. Further, the generating of the updated request comprises continuing an attempt to transmit undelivered work until configured criteria are met.

Further, in some embodiments, the connector is configured for collecting documents in a prescribed format for delivering a specific type of the at least one delivery device. Further, the connector is configured for transmitting to the at least one delivery device using a configuration provided in the updated request and the prepared delivery information. Further, the connector is configured for sending a status back to a delivery processor.

Further, in some embodiments, the receiving of the at least one recipient information comprises receiving recipient demographics. Further, the receiving of the at least one recipient information comprises updating a delivery device for a recipient, wherein the updating comprises adding the delivery device for the recipient and validating device changes.

In further embodiments, the method 2300 may include a step of receiving, using the communication device, at least one system configuration. Further, the receiving of the at least one system configuration comprises receiving a system configuration and either updating or adding the system configuration. Further, the receiving of the at least one system configuration comprises managing configurations such as a delivery transmission configuration.

In further embodiments, the method 2300 may include a step of connector preparing, using the processing device, a complete detail delivery device specific version of the queued item to be delivered to the specific recipient's EMR, Electronic delivery device, etc.

In further embodiments, the method 2300 may include a step of connector preparing, using the processing device, document image delivery, of the queued item to be delivered to the specific non-electronic repository delivery device, Fax, email, etc.

In further embodiments, the method 2300 may include a step of connector processing document delivery image, of the delivery version of the item to be delivered to an image delivery device, Fax, email, etc.

In further embodiments, the method 2300 may include a step of connector processing document delivery detail, of the delivery version of the item to be delivered to an electronic delivery device, EMR, electronic repository, etc.

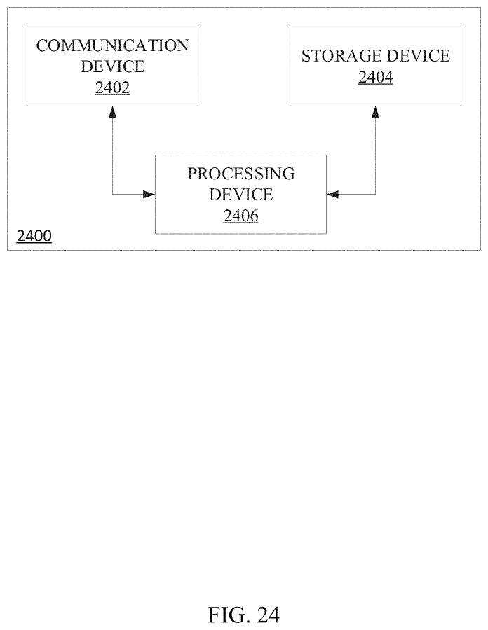

FIG. 24 is a block diagram of a system 2400 for facilitating the exchange of data, especially medical data associated with a patient, in accordance with some embodiments. The system 2400 may include a communication device 2402, a processing device 2404, and a storage device 2406. Further, the communication device 2402 may be configured for receiving a request (ROI) from at least one recipient device (recipient that makes the request). Further, the ROI may include a written signed request faxed to the IO. Further, the at least one recipient device may be associated with at least one recipient that may include a primary care provider, insurance companies, attorneys, courts, etc. Further, the at least one recipient device may include the recipient's Electronic Medical Record (EMR), fax, smartphone, tablet, laptop, etc. Further, the request may include the recipient's data associated with the at least one recipient. Further, the recipient's data may include name, email address, fax number, and so on. Further, the request may include information associated with the medical data of a patient that the at least one recipient may want to receive. Further, the recipient may want to receive the medical data in a data format corresponding to the at least one recipient device. Further, the medical data may include PHI, PII, medical documents, etc. Further, the communication device 2402 may be configured for transmitting a deliverable medical data to at least one healthcare entity device. Further, the deliverable medical data may include an information order to transmit the deliverable medical data to the at least one recipient device. Further, a single Release may be simultaneously delivered to multiple Recipients using their configured delivery device. Further, the communication device 2402 may be configured for receiving the medical data in a source data format from the at least one healthcare entity device. Further, the communication device 2402 may be configured for transmitting the deliverable medical data to the at least one recipient device. Further, transmitting the deliverable medical data may be based on medical regulatory act (or law) guidelines associated with a medical regulatory act (or law). Further, the medical regulatory act (or law) may include Health Insurance Portability and Accountability Act (HIPPA). Further, the communication device 2402 may be configured for receiving a delivery receipt from the at least one recipient device.

Further, the processing device 2404 may be communicatively coupled with the communication device 2402. Further, the processing device 2404 may be configured for analyzing the request to generate the deliverable medical data. Further, the processing device 2404 may be configured for processing the medical data to generate the deliverable medical data corresponding to a Recipient's data format.

Further, the storage device 2406 may be communicatively coupled with the processing device 2404. Further, the storage device 2406 may be configured for storing at least one of the medical data and the request. Further, at least one of the medical data and the request may be deleted based on the receiving of the delivery receipt. Further, in an embodiment, the medical data format may be identical to the deliverable data format.

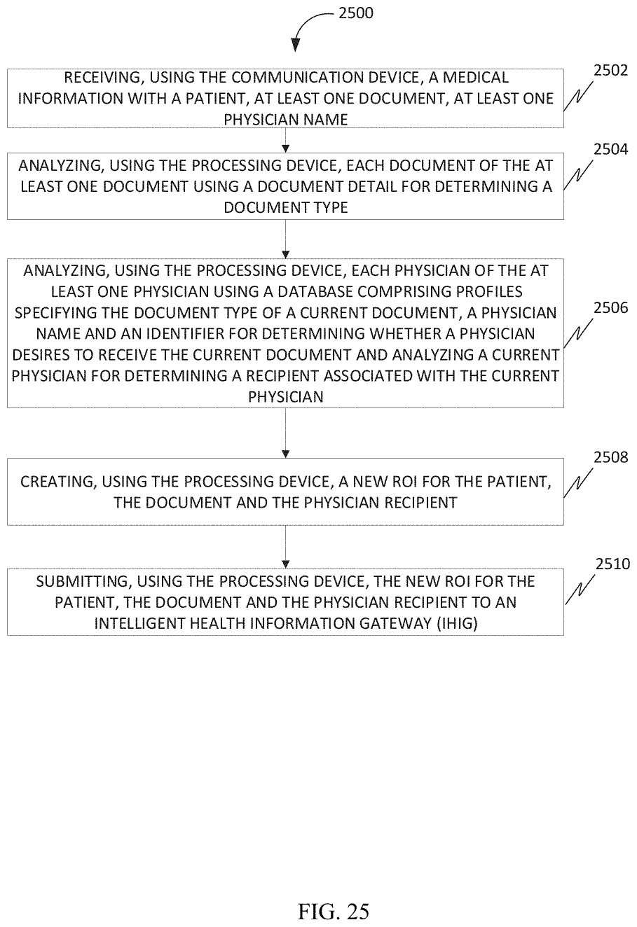

FIG. 25 is a flowchart of a method 2500 for facilitating automatic delivery of ROI, in accordance with some embodiments.

Accordingly, at 2502, the method 2500 may include receiving, using the communication device, a medical information with a patient, at least one document, at least one physician name.

Further, at 2504, the method 2500 may include analyzing, using the processing device, each document of the at least one document using a document detail for determining a document type.

Further, at 2506, the method 2500 may include analyzing, using the processing device, each physician of the at least one physician using a database comprising profiles specifying the document type of a current document, a physician name and an identifier for determining whether a physician desires to receive the current document and analyzing a current physician for determining a recipient associated with the current physician.

Further, at 2508, the method 2500 may include creating, using the processing device, a new ROI for the patient, the document and the physician recipient.

Further, at 2510, the method 2500 may include submitting, using the processing device, the new ROI for the patient, the document and the physician recipient to an Intelligent Health Information Gateway (IHIG). Further, the new ROI for the patient, the document and the physician recipient are submitted to the Intelligent Health Information Gateway (IHIG) as an IO. Further, one or more steps of the method 2500 may be repeated for each physician and the current document until done. Further, the one or more steps of the method 2500 may be repeated for each document until done.

FIG. 26 is a block diagram of a system 2600 showing the basic functionality of a connector used as a pluggable ingress, shown with dash lines, or final device delivery, shown with solid lines, processes. It may also be used where ever an easily configurable and buildable process is required. Further, the system 2600 may include a database (15 of FIG. 18) (2602), a connector config (2604), a gateway controller (16 of FIG. 18) (2606), a delivery controller (27 of FIG. 18) (2608), other process 2610, a pluggable connector 2612, a sending system (5 of FIG. 18) (2614), and a receiving system (28 of FIG. 18) (2616).

Accordingly, on startup of a connector (2600) the Pluggable Connector (2612) is the first thing started and is the main process loop continually waiting for work and processing each request upon receipt. This description of the Pluggable Connector 2612 will describe one that is the final delivery process of a release to a Recipient using the their Allscripts TouchWorks EMR implementing (20 of FIG. 18). After the Delivery Controller (27 of FIG. 18) (2608) finishes all the work necessary to provide the common delivery version of an ROI, it sends to the Pluggable Connector (2612) a message containing the complete common delivery version of the ROI appended to a complete AllScripts configuration including security and connection for this particular Recipient. Upon receipt of the message, the Pluggable Connector 2612 splits the configuration out from the message and the ROI is validated that the images for all the documents required have a URL of the actual image. Each image is requested using the URL and saved until needed for actual delivery. Once that is done, the Recipient's AllScripts server is logged into using the connection information and credentials from the provided configuration. With a valid login and session the patient in Allscripts is looked up using the patient information from the ROI by the fields defined in the configuration. If not found error, otherwise retrieve the first document's information in the list and retrieve the actual delivery image from the storage and tell AllScripts the document type of the upload and then upload the document capturing any errors, if none the document is marked as delivered. Repeat the upload process for each document in the list. The return status of each document back to the caller Delivery Controller (2608).

### Automatic Delivery

Automatic Delivery (AD) allows an IO to submit one or more documents for a patient along with a list of possible Recipients related to the patient the document is for and each document will be delivered to the Recipients who have indicated they would like to automatically receive the type of document (determined by the IO) submitted. Alternatively, if the IO has specified on the present disclosure's initial configuration that an AD will deliver the submitted document to all Recipients using the method specified in the submission or that specified in the physician's profile, default.

Step 1. An Automatic delivery message is received via an ingress using a GRPC, Restful, message queue, or similar connection FIG. 18 process 8. This message will provide the patient information that the Documents are, usually one related to, a list of the Documents (PDF, TIFF, FHIR records, etc.), usually one to be delivered, a list of the Recipients (Physicians, . . . ) who are associated with the patient who is to possibly receive the Documents and may contain other information specified by the IO that will be logged. The amount of detail provided is IO and implementation dependent, full detail, or just enough to allow the implementer to retrieve the information from the M.

Step 2. If the message only provided a minimal amount of information, the rest of the information is retrieved from the IO using a Connector as in FIG. 26 which may be configured for either the IO or the IO's EMR Vendor.

Step 3. Retrieve all document images. Each Document in the AD message may be in one of the multiple formats. The first is the IO's internal reference number or a URL to identify the Document. In this case, the connector for the IO is called to download the document's image and retrieve any other related information. In another case, the Document Image is provided directly. Other ways formats may be provided depending upon the implementation and are managed by Connectors. For each Document in the AD message, retrieve the image from the OI if required. Using Document Service 4003 (FIG. 21, save the Document providing the image and type of document and other document information provided in the AD message related to the Document. Save the ID of the new document and the Document Type in an array of all documents in the AD message to be used when creating the release for a Recipient.

Step 4. Retrieve patient information from the message as required for logging purposes and to retrieve an AD by the patient. The minimum patient information is their Medical Record Number, First Name, Last Name, SSN, and Date of Birth. Add Patient information to the Release Request.

Step 5. Validate each Recipient and create the Release Request specifically for each Recipient. The message may contain multiple recipients and each recipient will get only the Documents whose data type matches one of the types they signed up to receive using the Admin GUI 26 (FIG. 18) and stored in Recipient Profile 15 (FIG. 18). For each Recipient contained in the AD message, a check is made to confirm that as a minimum, the IO's unique ID for the Recipient plus any other IO specific information required to find the recipient in the IO system using a Connector. If the message does not contain all the requirements to identify the Recipient, a connector is used to connect to the IO and retrieve everything needed, and the Recipient Profile 15 (FIG. 18) is queried. If the required information is not available or after searching the Recipient Profile 15 (FIG. 18), a Recipient is not found, whatever Recipient information available is added to the Release Release Request along with the Status of FAILED, the Reason set to “Recipient Failed” and Step 5 is repeated for the next Recipient. Otherwise, continue with Step 6.

Step 5.1. Create the Release Request by using 4004 in FIG. 21. This Release Request is a place to store all the information related to one release for one patient to one Recipient delivering the documents the Recipient has signed up to receive in the Recipient Profile 15 (FIG. 18) defined in the AD. Once created it is built up in steps below.

Step 5.2. The message may contain multiple recipients and each recipient will get only the Documents whose data type matches one of the types they signed up to receive using the Admin GUI 26 (FIG. 18) and stored in Recipient Profile 15 (FIG. 18). For each Recipient contained in the AD message, a check is made to confirm that as a minimum, the IO's unique ID for the Recipient plus any other IO specific information required to find the recipient in the IO system using a Connector. If the message contains all the requirements to identify the Recipient, a connector is used to connect to the IO and retrieve everything needed, and the Recipient Profile 15 (FIG. 18) is queried. If the required information is not available or after searching the Recipient Profile 15 (FIG. 18), a Recipient is not found, whatever Recipient information available is added to the Release Request along with the Status of FAILED, the Reason set to “Recipient Failed” and Step 5.1 is repeated for the next Recipient. Otherwise, continue with Step 6.

Step 6. Add the documents from the document list (created in Step 4) of the types desired by the Recipient to the Release Request using 4004 of FIG. 21.

Step 7. Submit the Release Request as any other Release Request received from the IO. After processing step 6, the Release Request is a complete release of all the desired Documents for the specified patient and the specified Recipient, the Submit Delivery Service 4004 of (FIG. 21) is used to start the actual delivery process providing the Release Request ID and the Delivery Device ID retrieved from the Recipient Profile in Step 5 above. Notifications may be returned to the IO based upon their profile. Repeat Step 5 to process the next Recipient.

### HIPAA Validation Process

The VALIDATION PROCESS is a critical part of the present disclosure helping it meet HIPAA requirements.

STEP 1. Recipient registers using component 26 (FIG. 18) their delivery information. Information for a device they want to use for delivery. This includes all fax numbers, email addresses, EMR (login information and other details required to connect to their EMR) and any other devices of any type.

STEP 2. The registering of a device triggers the present disclosure to create a special validation release for that device. This release contains one document with a unique identifier and a URL.

STEP 3. For Fax, Email, and any non-electronic repository delivery the test release (step 1 above) will be sent to the delivery address. Upon receipt of the release, the recipient needs to open the URL provided they will have to enter the code and the secret question they entered during the setup. Once validated, there will be a form for them to complete the demographic collection for their profile. Once this is done that device is available to use and all releases pending because of non-validated delivery device are release for delivery.

STEP 4. Electronic direct to Requestor's EMR. This process will be required based upon the IO's requirements.

### Aspects

A first aspect of the healthcare entity includes at least one medical record repository containing the medical records for each patient who has received care at that facility or records received from other healthcare entities to allow for releasing medical records for a specific patient. A data processing system includes an EMR (Electronic Medical Record) or other system capable of processing a Release of Information (ROI) and submitting it for delivery to the specified recipient, and a cloud, on-premises, or hybrid implementation running an embodiment of this disclosed patent (IHIDS) comprising the steps of:
(A) receiving EMR provides the release basic information required for delivery via IHIDS;
(B) EMR provides each document required to be delivered via IHIDS:
(C) EMR provides recipient information for the delivery via IHIDS;
(D) EMR provides the delivery device to be delivered to via IHIDS;
(E) EMR submits the assembled release for delivery via IHIDS;
(F) EMR handles the release detail from IHIDS;
(G) EMR manages the delivery failure or success status;
(H) HIS (Hospital Information System) submits Automatic delivery to IHIDS;
(I) EMR submits a new recipient to IHIDS
(J) EMR accepts a new or updated recipient for IHIDS; and
(K) EMR accepts an electronic copy of anl(a) ROI from IHIDS.

A second aspect that is based on the method of the first aspect comprising the steps of providing a plugin application programming interface (API) each supporting one or more ingress methods (GRPC, HTTP-Restful, Message Queues, . . . ); and capable of executing the steps (A) through (G).

A third aspect that is based on the method of the second aspect includes the step of receiving the basic information of the release comprises the ingress creating a new release in a database, returning the new release ID back to the caller.

A fourth aspect that is based on the method of the second aspect includes the step of the ingress receiving each document contained in the release and saving it in a database. The source optionally may send the metadata and the URL of the document (Option 1) or the metadata and the actual full image of the document (Option 2).

A fifth aspect that is based on the method of the fourth aspect includes the source sending just the metadata and the URL of the image and saving the metadata in the database as a document and updating the Release of the new document. Returning the new document id.

A sixth aspect that is also based on the method of the fifth aspect includes the source sending both the metadata and the image, the image is saved in an image repository returning the id of the image then the metadata is saved along with the image id in a database, and then returning the new document id.

A seventh aspect that is based on the method of the second aspect includes the step of the ingress processing receipt of a recipient then confirming it is already in the database using the source's ID and retrieving the internal ID. If it, then its internal ID and source ID are added to the release. If it is not, the source's ID only is added to the release, and the internal is left blank.

An eighth aspect that is based on the method of the second aspect includes the step of the ingress processing receipt of a delivery device and confirming it is in the database and if it is not generic like “batchprint”, confirm that it belongs to the recipient.

A ninth aspect that is based on the method of the eighth aspect includes, further if the delivery device is generic or belongs to the recipient, it is added to the release. If it does not belong to the recipient add it to the release with an indicator prefixed to it.

A tenth aspect that is based on the method of the second aspect includes the step of the ingress processing a submit instruction by looking up the release in the database and creating a new delivery record from and sending the Delivery Controller a message to start the delivery process.

An eleventh aspect that is based on the method of the tenth aspect includes the steps of starting the delivery process: create a standard message to be used by all processes in delivery; determine the device to be used to deliver the Release and use the proper delivery connector for the specified delivery device; contact core requesting the requested release; retrieve all the document images for the release; concatenate the images into one image; retrieve any additional required information from the HIS, and queue the delivery to the proper delivery connector.

A twelfth aspect that is based on the method of the second aspect includes the step of the ingress processing an add recipient message, wherein the message received will contain all the demographic details the EMR has added to its recipient database plus the details of the delivery device(s) added for the recipient, further comprising the following steps: a.) determine if the recipient is already in the IHIDS database; b.) if it is, confirm the information matches; c.) if not, add both the recipient and the delivery device; and d.) release any pending deliveries.

A thirteenth aspect that is also based on the method of the second aspect includes the steps of the ingress processing and update recipient message, wherein the message received will contain all the demographic details the EMR has added to its recipient database plus the details of the delivery device(s) added for the recipient, further comprising the following steps: a.) determine if the recipient is in the IHIDS database; b.) if it is, updates the IHIDS database to match the EMR's; c.) if not, add both the recipient and the delivery device; and d.) send an update notification to the recipient.

According to some embodiments, a method for facilitating delivering of medical information of patients to a requestor's device is disclosed. Accordingly, the method may include a step of receiving, using a communication device, at least one request comprising at least one medical information of at least one patient from at least one source device. Further, only one request is processed at a time per process running. Further, any number of requests may be submitted, and all handled at the same time by multiple services, though one service can process one request at a time. Further, the at least one request may include a ROI, an interaction request, etc.

Further, the method may include a step of analyzing, using a processing device, the at least one request using at least one defined criteria of the implementation. Further, the analyzing of the at least one request allows distinguishing between the ROI request and the interaction request.

Further, the method may include a step of identifying, using the processing device, at least one destination device based on the analyzing of the at least one request, wherein the identifying of the at least one destination device comprises identifying at least one delivery device.

Further, the method may include a step of receiving, using the communication device, an unprocessed request of the at least one medical information associated with the at least one patient from the at least one source device based on the identifying of the at least one destination device.

Further, the method may include a step of determining, using the processing device, at least one delivery criterion based on the analyzing of the at least one request.

Further, the method may include a step of generating, using the processing device, an updated request of the at least one medical information associated with the at least one patient based on the unprocessed request of the at least one medical information and the at least one delivery criterion, wherein the updated request comprises a processed request.

Further, the method may include a step of transmitting, using the communication device, the processed request of the at least one medical information to the at least one destination device.

Further, the method may include a step of receiving, using the communication device, at least one recipient information comprising demographics and the at least one delivery device from the at least one source device.

Further, the method may include a step of storing, using a storage device, a validated version of the at least one recipient information in a database.

Further, the method may include a step of receiving, using the communication device, at least one medical information of patients with at least one recipient from the at least one source device.

Further, the method may include a step of generating using the processing device, a request containing the at least one medical information and at least one destination.

Further, in some embodiments, the method may include a step of determining, using the processing device, required information for delivery is comprised in the unprocessed request. Further, the method may include a step of extracting, using the storage device, various segments in the unprocessed request, wherein the various segments are stored. Further, the method may include a step of analyzing, using the processing device, the segments comprised in the unprocessed request.

Further, in an embodiment, the method may include a step of analyzing, using the processing device, the extracted segments using at least one machine learning model.

Further, in some embodiment, the identifying of the at least one delivery device comprises validating that the at least one delivery device is proper. Further, the identifying of the at least one delivery device comprises validating that the at least one delivery device is active. Further, the method may include a step of storing, using the storage device, a status of the at least one delivery device.

Further, in some embodiments, the determining of the at least one delivery criterion comprises validating an electronic medical record system defined is configured properly. Further, the determining of the at least one delivery criterion comprises validating other delivery devices defined is validated.

Further, in some embodiments, the generating of the updated request comprises creating the updated request with confirmed sub elements, wherein the confirmed sub elements comprise one or more of the at least one delivery device and the at least one recipient. Further, the generating of the updated request comprises transmitting the update request to a delivery connector for the specified delivery device type. Further, the generating of the updated request comprises receiving a delivery status from the delivery connector. Further, the generating of the updated request comprises continuing an attempt to transmit undelivered work until configured criteria are met.

Further, in some embodiments, the updated request of the at least one medical information to the at least one destination device receives a new work comprising receiving the updated request using a connector, collecting documents in a prescribed format for the at least one delivery device, transmitting to the at least one delivery device using a configuration provided in the updated request, and sending a status back to a delivery processor.

Further, in some embodiments, the receiving of the at least one recipient information comprises receiving recipient demographics. Further, the receiving of the at least one recipient information comprises updating a delivery device for a recipient, wherein the updating comprises adding the delivery device for the recipient. Further, the receiving of the at least one recipient information comprises validating device changes automatically.

Further, in some embodiments, the method may include a step of receiving, using the communication device, at least one system configuration, wherein the receiving of the at least one system configuration comprises receiving a system configuration and either updating or adding the system configuration. Further, the receiving of the at least one system configuration comprises managing configurations such as a delivery transmission configuration.

Further, the method may include verification processes. Further, the method may be associated with system configurations management environment.

According to some embodiments, a method for facilitating delivering of medical information of patients is disclosed. Accordingly, the method may include a step of receiving, using a communication device, an ingress connection for receiving a medical information of a patient from a source device. Further, the method may include a step of receiving, using the communication device, an unprocessed version of the medical information associated with the patient from the source device using the ingress connection. Further, the method may include a step of analyzing, using a processing device, the unprocessed version of the medical information using at least one dynamic configuration. Further, the method may include a step of identifying, using the processing device, at least one delivery device based on the analyzing of the unprocessed version of the medical information. Further, the method may include a step of generating, using the processing device, a delivery version of the medical information associated with the patient based on the unprocessed version of the medical information. Further, document URLs, document images, devices, and recipients are all separated out in the delivery version of the one or more medical information. Further, the method may include a step of transmitting, using the communication device, the delivery version of the medical information to a connector, wherein the connector is configured for creating a device specific version of the medical information based on the delivery version of the medical information for delivering the device specific version of the medical information to a specific type of the at least one delivery device. Further, the device specific version is created by the connector. Further, the device specific version knows the exact format the delivery device requires. (Eg FAX delivery requires the separate documents requested in the ROI to be concatenated into one lane image to be FAXed. The connector is the last point to collect the actual document images to be delivered. The only requirement for images on the unprocessed version is there is a document Image URL and possibly an actual document image. If there is no image when the connector receives the unprocessed version, it uses another connector to retrieve the images from the IO and stores them in an image repository. Further, steps in the IO submitting is contacting the ingress is open the URL for the proper ingress using the proper syntax for the ingress. Submitting the formatted release of information and closing the connection. The ingress receives it and submits it for processing that is discussed related to being the unprocessed version of the medical information. The connector is responsible for preparing the deliverable version to the device specific version and making sure that all the images are in the local repository and retrieving any that are missing. Then splitting things up as necessary for delivery processing, For FAX all documents in the ROI are concatenated into one large document. Processing, connector to the delivery device or other delivery service that will ultimately deliver to the delivery device (email, fax servers) and following the device specific rules of how to deliver each report places the document in the correct patient's proper folder for the type of report being processed following the rules for delivering directly to the Recipients EMR or instructing the intermediate delivery device for FAX, EMAIL . . . as to what to send and to where.

Further, in some embodiments, the dynamic configurations are set to the various things that use it on the call to them. An example of a powerful use of dynamic configurations is an email connector receiving the dynamic configuration to deliver an email. In this case, the connector is written to support multiple email delivery servers, each requiring different configurations. This is done with a code designed to take the dynamic configuration provided with the message to deliver an email. The connector knows how to use that new information to complete the process, configuring itself to be an interface to that specific email server.

Further, in some embodiments, the connector is configured for determining at least one delivery method for delivering the device specific version of the medical information based on the delivery version of the medical information, wherein the at least one delivery device is associated with at least one recipient, wherein the device specific version of the medical information is delivered using the at least one delivery method.

Further, in an embodiment, the at least one delivery method comprises a delivery to electronic medical record (EMR) method, wherein the device specific version of the medical information is delivered to an electronic medical record (EMR) associated with the patient in the delivery to EMR method.

Further, in some embodiments, the connector is configured for determining at least one delivery criterion associated with the delivery version of the medical information, wherein the at least one delivery criterion comprises at least one information format for the medical information, wherein the connector is configured for formatting the delivery version of the medical information using the at least one information format, wherein the creating of the device specific version of the medical information is further is based on the formatting.

In further embodiments, the method may include a step of accessing, using the processing device, the at least one source device, wherein the at least one source device comprises the unprocessed version of the medical information of the patient, wherein the receiving of the unprocessed version of the medical information from the at least one source device is further based on the accessing.

In further embodiments, the method may include a step of analyzing, using the processing device, the unprocessed version of the medical information using the at least one dynamic configuration. Further, the method may include a step of determining, using the processing device, at least one delivery condition for the delivering of the medical information based on the analyzing of the unprocessed version of the medical information, wherein the generating of the delivery version of the medical information is further based on the determining of the at least one delivery condition, wherein the delivery version of the medical information comprises the at least one delivery condition.

In further embodiments, the method may include a step of determining, using the processing device, a requirement of at least one additional information associated with the patient based on the analyzing the unprocessed version of the medical information. Further, the method may include a step of retrieving, using a storage device, the at least one additional information associated with the patient from a database based on the determining of the requirement, wherein the generating of the delivery version of the medical information is further based on the at least one additional information.

According to some embodiments, a system for facilitating delivering of medical information of patients is disclosed. Accordingly, the system may include a communication device and a processing device. Further, the communication device may be configured for receiving an ingress connection for receiving a medical information of a patient from a source device. Further, the communication device may be configured for receiving an unprocessed version of the medical information associated with the patient from the source device using the ingress connection. Further, the communication device may be configured for transmitting a delivery version of the medical information to a connector, wherein the connector is configured for creating a device specific version of the medical information based on the delivery version of the medical information for delivering the device specific version of the medical information to a specific type of the at least one delivery device. Further, the processing device may be communicatively coupled with the communication device. Further, the processing device is configured for analyzing the unprocessed version of the medical information using at least one dynamic configuration. Further, the processing device is configured for identifying at least one delivery device based on the analyzing of the unprocessed version of the medical information. Further, the processing device is configured for generating the delivery version of the medical information associated with the patient based on the unprocessed version of the medical information.

Further, in some embodiments, the connector is configured for determining at least one delivery method for delivering the device specific version of the medical information based on the delivery version of the medical information, wherein the at least one delivery device is associated with at least one recipient, wherein the device specific version of the medical information is delivered using the at least one delivery method.

Further, in an embodiment, the at least one delivery method comprises a delivery to electronic medical record (EMR) method, wherein the device specific version of the medical information is delivered to an electronic medical record (EMR) associated with the patient in the delivery to EMR method.

Further, in some embodiments, the connector is configured for determining at least one delivery criterion associated with the delivery version of the medical information, wherein the at least one delivery criterion comprises at least one information format for the medical information, wherein the connector is configured for formatting the delivery version of the medical information using the at least one information format, wherein the creating of the device specific version of the medical information is further is based on the formatting.

Further, in some embodiments, the processing device is further configured for accessing the at least one source device, wherein the at least one source device comprises the unprocessed version of the medical information of the patient, wherein the receiving of the unprocessed version of the medical information from the at least one source device is further based on the accessing.

Further, in some embodiments, the processing device is configured for analyzing the unprocessed version of the medical information using the at least one dynamic configuration. Further, the processing device is configured for determining at least one delivery condition for the delivering of the medical information based on the analyzing of the unprocessed version of the medical information, wherein the generating of the delivery version of the medical information is further based on the determining of the at least one delivery condition, wherein the delivery version of the medical information comprises the at least one delivery condition.

Further, in some embodiments, the processing device is further configured for determining a requirement of at least one additional information associated with the patient based on the analyzing the unprocessed version of the medical information, wherein the processing device is communicatively coupled with a storage device, wherein the storage device is configured for retrieving the at least one additional information associated with the patient from a database based on the determining of the requirement, wherein the generating of the delivery version of the medical information is further based on the at least one additional information.

Although the present disclosure has been explained in relation to its preferred embodiment, it is to be understood that many other possible modifications and variations can be made without departing from the spirit and scope of the disclosure.

What is claimed is:

1. A method for facilitating delivering of medical information of patients to a requestor's device, the method comprising:
receiving, using a communication device, at least one request comprising at least one medical information of at least one patient from at least one source device;
analyzing, using a processing device, the at least one request using at least one defined criteria of the implementation;
identifying, using the processing device, at least one destination device based on the analyzing of the at least one request, wherein the identifying of the at least one destination device comprises identifying at least one delivery device;
receiving, using the communication device, an unprocessed request of the at least one medical information associated with the at least one patient from the at least one source device;
determining, using the processing device, at least one delivery criterion based on the analyzing of the at least one request;
generating, using the processing device, an updated request of the at least one medical information associated with the at least one patient based on the unprocessed request of the at least one medical information and the at least one delivery criterion, wherein the updated request comprises an interim request;
transmitting, using the communication device, the interim request of the at least one medical information to a connector;
receiving, using the communication device, at least one recipient information comprising demographics and the at least one delivery device from the at least one source device;
storing, using a storage device, a validated version of the at least one recipient information in a database;
receiving, using the communication device, at least one medical information of patients with at least one recipient from the at least one source device;
generating using the processing device, a request containing the at least one medical information and at least one destination; and
transmitting, using the communication device, the request to a connector.

2. The method of claim 1 further comprising:
receiving, using the communication device, a medical information with a patient, at least one document, at least one physician name;
analyzing, using the processing device, each document of the at least one document using a document detail for determining a document type;
analyzing, using the processing device, each physician of the at least one physician using a database comprising profiles specifying the document type of a current document, a physician name and an identifier for determining whether a physician desires to receive the current document and analyzing a current physician for determining a recipient associated with the current physician;
creating, using the processing device, a new ROI for the patient, the document and the physician recipient; and
submitting, using the processing device, the new ROI for the patient, the document and the physician recipient to an Intelligent Health Information Gateway (IHIG).

3. The method of claim 1 further comprising:
determining, using the processing device, whether required information for delivery is comprised in the unprocessed request;
extracting, using the storage device, various segments from the unprocessed request, wherein the various segments are stored; and
analyzing, using the processing device, the segments comprised in the unprocessed request.

4. The method of claim 3 further comprising analyzing, using the processing device, the extracted segments using at least one dynamic configuration.

5. The method of claim 1, wherein the identifying of the at least one delivery device comprises:
validating that the at least one delivery device is proper; and
validating that the at least one delivery device is active, wherein the method further comprises storing, using the storage device, a status of the at least one delivery device.

6. The method of claim 1, wherein the determining of the at least one delivery criterion comprises:
validating whether an electronic medical record system is configured properly; and
validating other delivery devices.

7. The method of claim 1, wherein the generating of the updated request comprises:
creating the updated request with confirmed sub elements, wherein the confirmed sub elements comprises one or more of the at least one delivery device, the at least one recipient, and at least one document to be delivered;
transmitting the update request to a delivery connector for a specified delivery device type;
receiving a delivery status from the delivery connector; and
continuing an attempt to transmit undelivered work until configured criteria are met.

8. The method of claim 1, wherein the connector is configured for:
collecting documents in a prescribed format for delivering a specific type of the at least one delivery device;
transmitting to the at least one delivery device using a configuration provided in the updated request and the prepared delivery information; and
sending a status back to a delivery processor.

9. The method of claim 1, wherein the receiving of the at least one recipient information comprises:
receiving recipient demographics;
updating a delivery device for a recipient, wherein the updating comprises adding the delivery device for the recipient; and
validating device changes.

10. The method of claim 1 further comprising receiving, using the communication device, at least one system configuration, wherein the receiving of the at least one system configuration comprises:
receiving a system configuration and either updating or adding the system configuration; and
managing configurations such as a delivery transmission configuration.

11. The method of claim 1 further comprising:
connector preparing, using the processing device, a complete detail delivery device specific version of a queued item to be delivered to at least one of a specific recipient's EMR and an Electronic delivery device.

12. The method of claim 1 further comprising:
connector preparing, using the processing device, document image delivery, of the queued item to be delivered to at least one of a specific non-electronic repository delivery device, a Fax, and an email.

13. The method of claim 1 further comprising:
connector processing document delivery image, of the delivery version of an item to be delivered to at least one of an image delivery device, a Fax, and an email.

14. The method of claim 1 further comprising:
connector processing document delivery detail, of the delivery version of an item to be delivered to at least one of an electronic delivery device, an EMR, and an electronic repository.

15. A method for facilitating delivering of medical information of patients, the method comprising:
receiving, using a communication device, an ingress connection for receiving a medical information of a patient from a source device;
receiving, using the communication device, an unprocessed version of the medical information associated with the patient from the source device using the ingress connection;
analyzing, using a processing device, the unprocessed version of the medical information using at least one dynamic configuration;
identifying, using the processing device, at least one delivery device based on the analyzing of the unprocessed version of the medical information;
generating, using the processing device, a delivery version of the medical information associated with the patient based on the unprocessed version of the medical information; and
transmitting, using the communication device, the delivery version of the medical information to a connector, wherein the connector is configured for creating a device specific version of the medical information based on the delivery version of the medical information for delivering the device specific version of the medical information to a specific type of the at least one delivery device.

16. The method of claim 15, wherein the connector is configured for determining at least one delivery method for delivering the device specific version of the medical information based on the delivery version of the medical information, wherein the at least one delivery device is associated with at least one recipient, wherein the device specific version of the medical information is delivered using the at least one delivery method.

17. The method of claim 16, wherein the at least one delivery method comprises a delivery to electronic medical record (EMR) method, wherein the device specific version of the medical information is delivered to an electronic medical record (EMR) associated with the patient in the delivery to EMR method.

18. The method of claim 15, wherein the connector is configured for determining at least one delivery criterion associated with the delivery version of the medical information, wherein the at least one delivery criterion comprises at least one information format for the medical information, wherein the connector is configured for formatting the delivery version of the medical information using the at least one information format, wherein the creating of the device specific version of the medical information is further is based on the formatting.

19. The method of claim 15 further comprising accessing, using the processing device, the at least one source device, wherein the at least one source device comprises the unprocessed version of the medical information of the patient, wherein the receiving of the unprocessed version of the medical information from the at least one source device is further based on the accessing.

20. The method of claim 15 further comprising:
analyzing, using the processing device, the unprocessed version of the medical information using the at least one dynamic configuration; and
determining, using the processing device, at least one delivery condition for the delivering of the medical information based on the analyzing of the unprocessed version of the medical information, wherein the generating of the delivery version of the medical information is further based on the determining of the at least one delivery condition, wherein the delivery version of the medical information comprises the at least one delivery condition.

21. The method of claim 15 further comprising:
determining, using the processing device, a requirement of at least one additional information associated with the patient based on the analyzing the unprocessed version of the medical information; and
retrieving, using a storage device, the at least one additional information associated with the patient from a database based on the determining of the requirement, wherein the generating of the delivery version of the medical information is further based on the at least one additional information.

22. A system for facilitating delivering of medical information of patients, the system comprising:
a communication device configured for:
receiving an ingress connection for receiving a medical information of a patient from a source device;
receiving an unprocessed version of the medical information associated with the patient from the source device using the ingress connection; and
transmitting a delivery version of the medical information to a connector, wherein the connector is configured for creating a device specific version of the medical information based on the delivery version of the medical information for delivering the device specific version of the medical information to a specific type of the at least one delivery device; and
a processing device communicatively coupled with the communication device, wherein the processing device is configured for:
analyzing the unprocessed version of the medical information using at least one dynamic configuration;
identifying at least one delivery device based on the analyzing of the unprocessed version of the medical information; and
generating the delivery version of the medical information associated with the patient based on the unprocessed version of the medical information.

23. The system of claim 22, wherein the connector is configured for determining at least one delivery method for delivering the device specific version of the medical information based on the delivery version of the medical information, wherein the at least one delivery device is associated with at least one recipient, wherein the device specific version of the medical information is delivered using the at least one delivery method.

24. The system of claim 23, wherein the at least one delivery method comprises a delivery to electronic medical record (EMR) method, wherein the device specific version of the medical information is delivered to an electronic medical record (EMR) associated with the patient in the delivery to EMR method.

25. The system of claim 22, wherein the connector is configured for determining at least one delivery criterion associated with the delivery version of the medical information, wherein the at least one delivery criterion comprises at least one information format for the medical information, wherein the connector is configured for formatting the delivery version of the medical information using the at least one information format, wherein the creating of the device specific version of the medical information is further is based on the formatting.

26. The system of claim 22, wherein the processing device is further configured for accessing the at least one source device, wherein the at least one source device comprises the unprocessed version of the medical information of the patient, wherein the receiving of the unprocessed version of the medical information from the at least one source device is further based on the accessing.

27. The system of claim 22, wherein the processing device is further configured for:
analyzing the unprocessed version of the medical information using the at least one dynamic configuration; and
determining at least one delivery condition for the delivering of the medical information based on the analyzing of the unprocessed version of the medical information, wherein the generating of the delivery version of the medical information is further based on the determining of the at least one delivery condition, wherein the delivery version of the medical information comprises the at least one delivery condition.

28. The system of claim 22, wherein the processing device is further configured for determining a requirement of at least one additional information associated with the patient based on the analyzing the unprocessed version of the medical information, wherein the processing device is communicatively coupled with a storage device, wherein the storage device is configured for retrieving the at least one additional information associated with the patient from a database based on the determining of the requirement, wherein the generating of the delivery version of the medical information is further based on the at least one additional information.
